(19) United States
(12) Patent Application Publication
(10) Pub. No.: US 2019/0046165 A1
(43) Pub. Date: Feb. 14, 2019

(54) Method and Device for Concentration of Cellular Suspensions

(71) Applicant: Wenzel Spine, Inc., Austin, TX (US)

(72) Inventors: Jeremy Lemoine, Leander, TX (US); Erik Emstad, Austin, TX (US); Michael Landry, Austin, TX (US)

(21) Appl. No.: 16/059,363

(22) Filed: Aug. 9, 2018

Related U.S. Application Data
(60) Provisional application No. 62/543,484, filed on Aug. 10, 2017.

(51) Int. Cl. A61B 10/02; A61B 10/00

(52) U.S. Cl. CPC A61B 10/025; A61B 10/0096; A61M 1/34; A61B 2010/0258; A61B 10/0283

(57) ABSTRACT

An embodiment includes a system comprising: a lower piston in a lower chamber and an upper piston in an upper chamber; a filter between the lower and upper pistons; at least one processor and memory to: move the lower piston away from the filter, while the upper piston is still, to draw bone marrow aspirate (BMA) into the lower chamber from an external container; move the lower piston towards the filter while simultaneously moving the upper piston away from the filter to advance BMA fluid of the BMA from the lower chamber across the filter and into the upper chamber and to locate BMA cells of the BMA adjacent the filter and within the lower chamber; move the lower piston toward the filter, while the upper piston is still, to advance the BMA cells out of the lower chamber and into one of the external container and another external container.

This application claims priority to U.S. Provisional Patent Application No. 62/543,484 filed on Aug. 10, 2017 and entitled “Method and Device for Concentration of Cellular Suspensions”, the content of which is hereby incorporated by reference.

### TECHNICAL FIELD

The present disclosure generally relates to medical systems and methods for separating heterogeneous suspensions of autologous cells into various fractions, such as platelet fractions or mononuclear cell fractions, for therapeutic point-of-care use. Specifically, an embodiment relates to methods and systems for the separation and concentration of mononuclear cell suspensions from whole bone marrow aspirates (BMA) or whole blood.

### BACKGROUND

Separation of heterogeneous cell suspensions such as marrow or blood may be done by centrifugation. Particles suspended in fluid are subject to two forces: frictional force from the fluid on the particles, which push the particles up and keep them from precipitating out of solution; and, a gravitational force, which pulls the particles down and out of solution. Centrifugation increases the force on all particles and induces their separation based on the particle's sedimentation velocity, radius (size), and the viscosity of the surrounding fluid. The sedimentation velocity for a given particle is directly proportional to the difference in the mass density of said particles and that of the fluid in which it is suspended. It is also directly proportional to the square of the radius of each particle.

Most systems for the production of mononuclear cells (MNC) or platelets from whole blood or BMA use centrifugation. Some use a single step process which captures the cells of interest inside a compartment within the centrifugation tube. Others use a two-step process whereby a significant portion of the red blood cells (RBC) are pelleted in the first centrifugation step, discarded, and the supernatant is spun a second time under higher force to pellet total nucleated cells (TNC) and platelets.

### BRIEF DESCRIPTION OF FIGURES

Features and advantages of embodiments of the present invention will become apparent from the appended claims, the following detailed description of one or more example embodiments, and the corresponding figures. Where considered appropriate, reference labels have been repeated among the figures to indicate corresponding or analogous elements.

FIG. 1 provides a cross-sectional view of an embodiment of a filter apparatus including dual, independently controlled pistons, a semi-permeable filter medium, and filter supports to prevent deflection of the filter media.

FIG. 2 provides an external view of an embodiment of the invention, depicting dual chambers and a framework to provide compression of the seal to prevent fluid escape.

FIG. 3a depicts the filter apparatus in an embodiment which includes one means of providing piston motion, stepper motors coupled to a threaded rod via drive belts and drive pulleys. FIG. 3b provides the same embodiment in an isometric view that allows better visualization of the motor and pulley configurations.

FIGS. 4a and 4b provide an isometric cross-sectional view of an embodiment of the device and a schematic for visualization, respectively. In both figures, the device is in the initial position, with pistons fully seated against the filter material, and engagement of the piston face protrusions into the void spaces of the filter support grates. No fluid has entered the device at this time.

FIGS. 5a and 5b provide an isometric cross-sectional view of an embodiment of the device and a schematic for visualization, respectively. In both figures, the device is in the intake position, with the intake side piston withdrawn to draw the unfiltered fluid with cells into the reservoir, while the dead end piston (so named because the fluid in this chamber is at a dead end, and cannot exit the chamber in any way other than that in which it entered, through the filter) remains fully seated against the filter material, and engagement of the piston face protrusions into the void spaces of the filter support grates.

FIGS. 6a and 6b provide an isometric cross-sectional view of an embodiment of the device and a schematic for visualization, respectively. In both figures, the device is in the filter position, where the intake side piston has been fully seated against the filter material, and engagement of the piston face protrusions into the void spaces of the filter support grates, while the dead end piston has been withdrawn to the draw fluid through the filter and into the fluid trap.

FIGS. 7a and 7b provide an isometric cross-sectional view of an embodiment of the device and a schematic for visualization, respectively. In both figures, the device is in the backflush position. The intake side piston has been partially withdrawn from the filter material while the dead end piston has been partially compressed to force a small portion of the fluid in the reverse direction across the filter material.

FIGS. 8a and 8b provide an isometric cross-sectional view of an embodiment of the device and a schematic for visualization, respectively. In both figures, the device is in the ejection position. The intake side piston has been fully seated against the filter material, and engagement of the piston face protrusions into the void spaces of the filter support grates, while the dead end piston retains the position it held at the backflush stage, causing an increase in pressure of the fluid, and inducing flow out of the system on the intake side.

FIGS. 9a and 9b provide an isometric cross-sectional view of an embodiment of the device and a schematic for visualization, respectively. In both figures, the device is in the buffer position. This is an alternative method for filtration. In this case, the intake side piston has been partially withdrawn to draw a fraction of the unfiltered fluid with cells into the reservoir to allow for distribution over the piston and allow full wetting of filter and filtration to occur over the entirety of the filter surface, while the dead end piston remains fully seated against the filter material, and engagement of the piston face protrusions into the void spaces of the filter support grates.

FIGS. 10a and 10b provide an isometric cross-sectional view of an embodiment of the device and a schematic for visualization, respectively. In both figures, the device is in the buffered filtration position. In this case, the intake side piston remains partially withdrawn to maintain a buffer space to allow for fluid flow that is not restricted as it would be when the intake piston is fully seated against the filter. The dead end piston has been withdrawn to pull the remainder of the fluid into the chambers and through the filter and into the fluid trap.

FIGS. 11a and 11b provide an isometric cross-sectional view of an embodiment of the device and a schematic for visualization, respectively. In both figures, the device is in the de-buffer position. In this case, the intake side piston has been fully seated against the filter material, and engagement of the piston face protrusions into the void spaces of the filter support grates, while the dead end piston has been withdrawn to pull the remaining buffer portion of the fluid through the filter and into the fluid trap. Ejection may then occur as described with FIGS. 8a and 8b.

FIG. 12 provides a cross-sectional view of the filter apparatus described with the addition of an extension on the piston that contacts the outer wall of the filtration chamber at a point distal to the filter media to prevent canting of the piston in the chamber.

FIG. 13a depicts an embodiment of a piston face, including protrusions that interface with the filter support grate. Additionally, the embodiment demonstrates a singular port, as would be used on the intake piston. In this case, one way valves control the flow into and out of the device through the same port. FIG. 13b depicts the same, in an isometric view for better visualization of the part depth.

FIG. 14a depicts the face of an embodiment of the filter support grate that operates with the pistons depicted in FIGS. 13a, 13b, 15a, 15b, 16a, 16b, 17a, and 17b. The filter support grate has voids that correspond to the protrusion on the face of the piston. FIG. 14b depicts the same, in an isometric view for better visualization of the part depth.

FIG. 15a depicts the face of an embodiment of the piston face, including protrusions that interface with the filter support grate. Additionally, the embodiment demonstrates dual ports, as would be used on the intake piston. In this case, one port allows for flow into the device, while another allows for flow out of the device. One way valves would still control the flow into and out of the device. FIG. 15b depicts the same, in an isometric view for better visualization of the part depth.

FIG. 16a depicts the face of an embodiment of the piston face, including protrusions that interface with the filter support grate. Additionally, the embodiment demonstrates multiple ports, as would be used on the intake piston. In this case, one port allows for flow into the device, while the other ports allow for flow out of the device. One way valves would still control the flow into and out of the device. A manifold would be utilized to join the output ports into one stream. FIG. 16b depicts the same, in an isometric view for better visualization of the part depth.

FIG. 17a depicts the face of an embodiment of the piston face, including protrusions that interface with the filter support grate. Additionally, the embodiment demonstrates multiple ports, as would be used on the intake piston. In this case, one port allows for flow into the device, while the other ports allow for flow out of the device. In this embodiment, the output ports are shown in the recesses of the piston face instead of the on the face of the protrusions. One way valves would still control the flow into and out of the device. A manifold would be utilized to join the output ports into one stream. FIG. 17b depicts the same, in an isometric view for better visualization of the part depth.

FIG. 18 depicts a method to complete the filtration process using the device depicted in the prior figures. The figure depicts the overall process of operation of the device, with the logic and functionality in a macroscopic view. Move loops are shown in the flow chart for each portion of the filtration and concentration process.

FIGS. 19a through 19g depict pseudocode utilized in a microcontroller to control a set of stepper motors as depicted in, for example, FIGS. 3a and 3b. The pseudocode references control of these motors, but in another embodiment, is modified to control pneumatic or hydraulic cylinders. The pseudocode also allows for communication to the end user via a touchscreen or signal LED, but in another embodiment can also include audible cues to the end user.

FIGS. 20-27 depict a system and method for concentration of cellular suspensions in an embodiment.

FIGS. 28-33 depict a system and method for concentration of cellular suspensions in an embodiment.

### DETAILED DESCRIPTION

In the following description, numerous specific details are set forth but embodiments of the invention may be practiced without these specific details. Well-known circuits, structures and techniques have not been shown in detail to avoid obscuring an understanding of this description. “An embodiment”, “various embodiments” and the like indicate embodiment(s) so described may include particular features, structures, or characteristics, but not every embodiment necessarily includes the particular features, structures, or characteristics. Some embodiments may have some, all, or none of the features described for other embodiments. “First”, “second”, “third” and the like describe a common object and indicate different instances of like objects are being referred to. Such adjectives do not imply objects so described must be in a given sequence, either temporally, spatially, in ranking, or in any other manner.

Applicant determined separation of a mixture of particles suspended in the same fluid using centrifugation alone is effective when there is a large difference in the mass density of the particles and their radius. However, for particles of similar size and density, centrifugal separation is less efficient, even with sequential centrifugation steps.

Applicant determined an undesirable component of MNC and platelet separation by centrifugation (at least in some situations) is the high concentration of RBC in the final concentrate. This high concentration of RBC is due, at least in part, to the above mentioned inefficiency.

Excess RBCs can elicit an undesirable inflammatory response and decrease the healing rate in sites with low perfusion by the accumulation of cellular debris and intracellular components released upon RBC lysis. The mass density of red blood cells changes with centrifugation and packing; however, the mass density is never significantly higher than the mass density of white blood cells (MNC). The mass density of MNC is 1040 kg/m3, the mass density of RBC pellets changes with its packing density so that a 100% preparation of RBC has a density of 1110 kg/m3, the density of a 90% preparation is 1090 kg/m3, and that of 70% is 1070 kg/m3. Due to this, Applicant determined the use of centrifugation alone for fractionating heterogeneous physiological cell mixtures requires a trade-off between MNC yield and RBC content. An increase in MNC yield forces an undesirable concentration of RBC in the same layer while eliminating RBC significantly decreases the yield of MNC.

In addition, Applicant determined centrifugation-based systems require the use of an apparatus capable of applying a centrifugal force no less than 1000×g. Using conventional technology, this requirement limits the point-of-care use of such devices within the operating room by impeding their use within the sterile field, forcing the BMA to leave the sterile field. This creates a risk for contamination and crowds the operating room by requiring floor space for the equipment.

However, an embodiment solves these issues by offering a compact, self-contained, sterile packaged device with a simplified interface for usage. The device is designed such that the bone marrow aspirate is maintained in a sealed portion of the device that does not allow contact with the mechanical mechanisms of the device. The device itself includes materials that are able to allow for sterilization through ethylene oxide or gamma radiation sterilization. The device can be opened in the sterile field and plugged into an outlet. The OR staff then connects the bone marrow aspirate syringe to the device using a standard connection, and then activates the machine (e.g., by a push-button). The device then enters an automated process to remove the red blood cells from the aspirate, and then to concentrate the remaining cellular content into a smaller volume of plasma, and eject this cell-rich component into another syringe which has been connected to the device.

As explained above, an embodiment eliminates (or at least reduces) the need for in-depth knowledge and user interaction with the device, eliminates the contamination threat of crossing the sterile barrier, reduces the space needed in the OR as it can be pulled off a shelf and utilized on an as needed basis, and then can be either discarded or recycled.

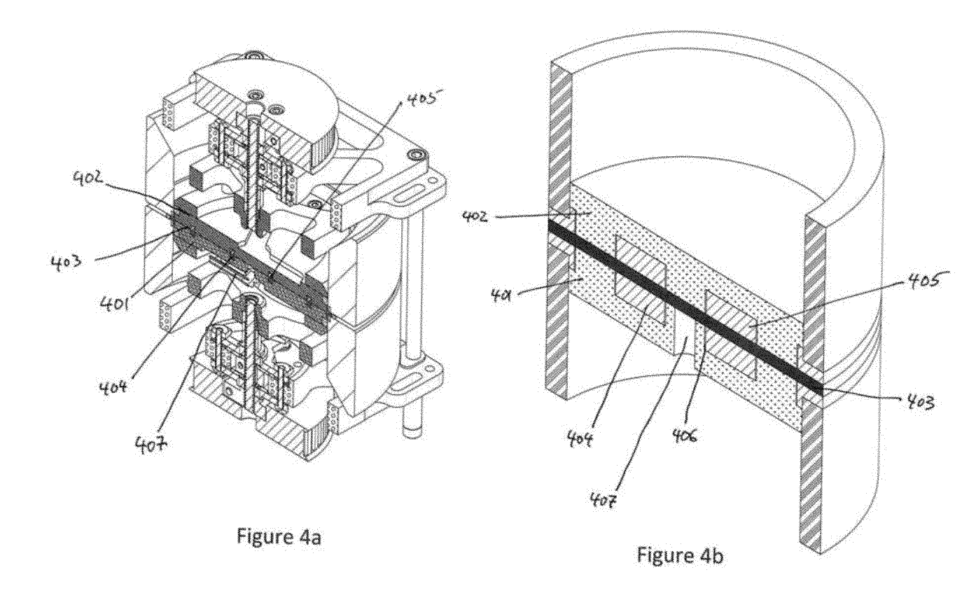

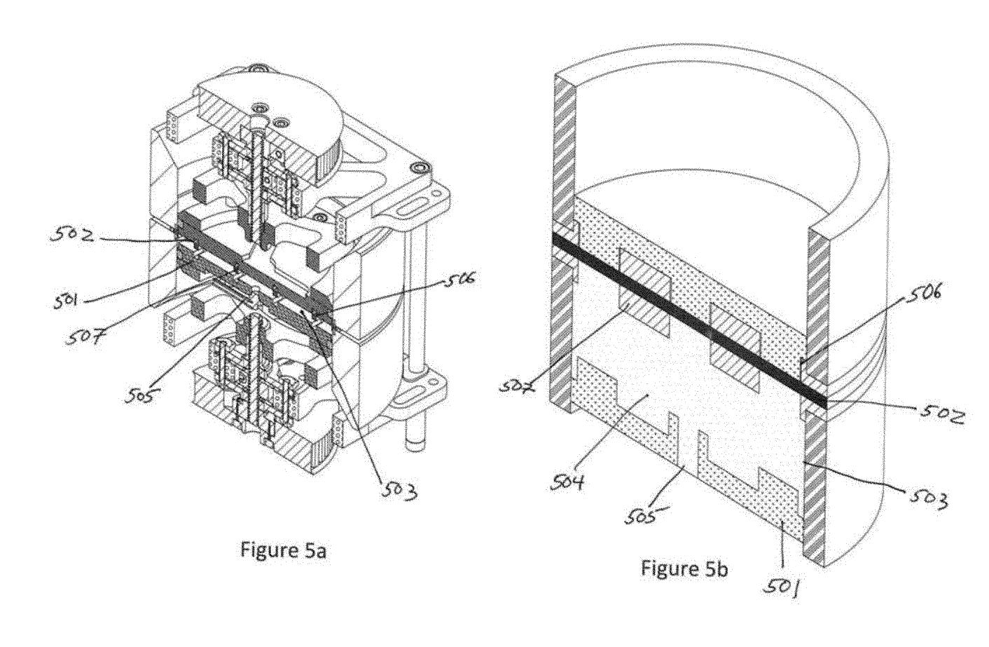

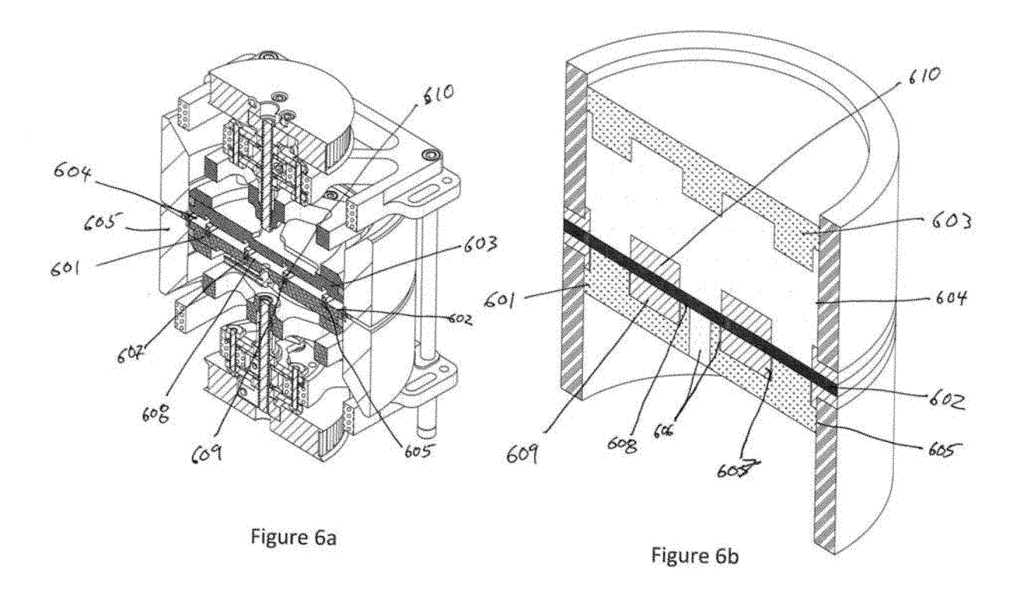

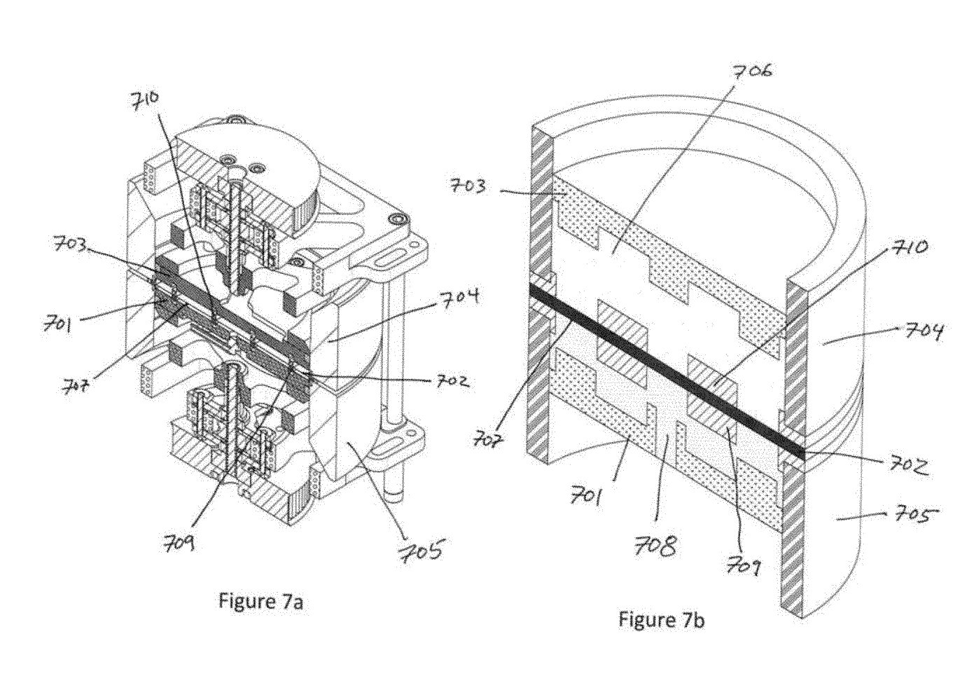

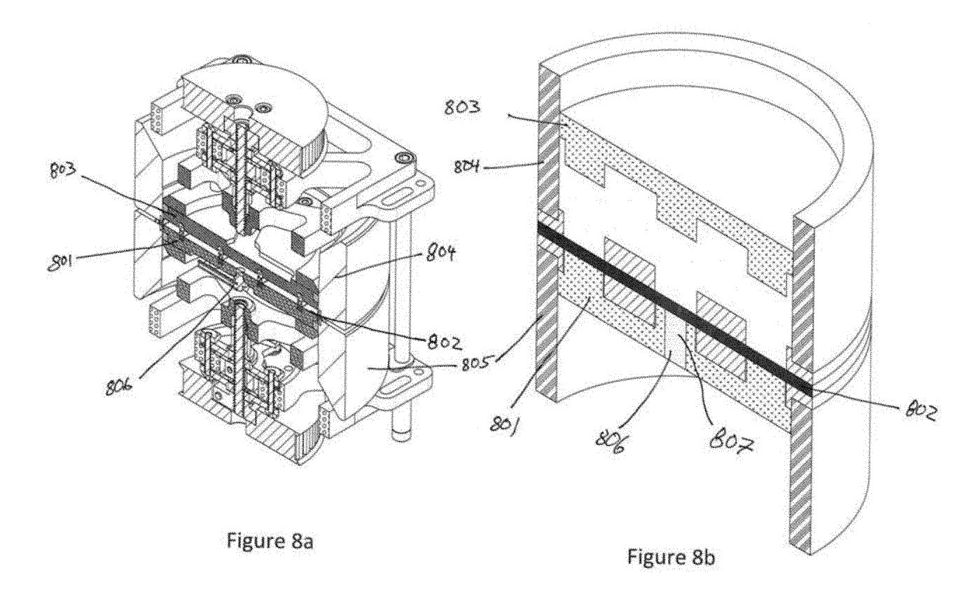

An embodiment functions via the following steps. Step (1) includes the intake of bone marrow aspirate. Step (2) includes removing RBCs or erythrocytes using one of any variety of methods, including low speed centrifugation, addition of a flocculent and allowing sedimentation, enhanced flow flocculation, and the like. Step (3) includes the reduced RBC plasma and cell suspension decanted or otherwise transferred to a chamber for concentration and filtration (see, e.g., FIG. 4B). Step (4) includes concentration/filtration as the fluid is forced through a semi-permeable membrane. This allows fluid to pass through the membrane, while trapping the cellular content and platelets on the inlet side of the membrane (see, e.g., FIGS. 5b and 6b). Step (5) includes a small backflush of the fluid component is applied to the filter to dislodge any cells and force them back into suspension (see, e.g., FIG. 7b). Step (6) includes the highly-concentrated cell solution is then output to a clean output syringe (connected to a separate syringe connection) that the user can disconnect and use as normal (see, e.g., FIG. 8b).

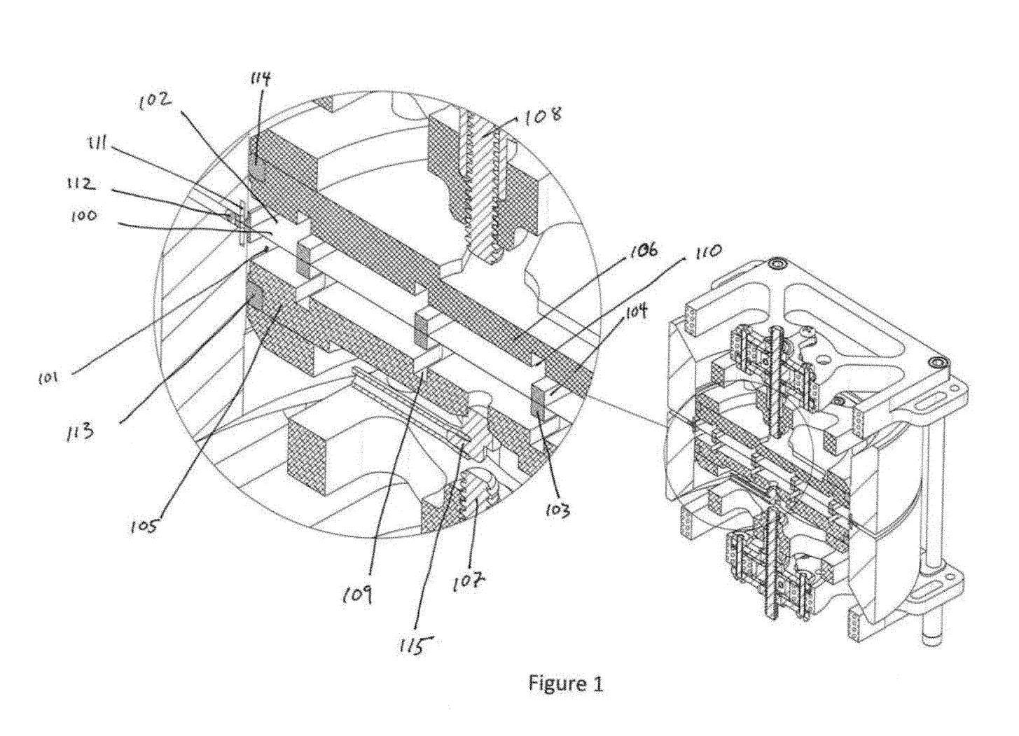

FIG. 1 demonstrates an embodiment of the filtration device in a sectioned isometric view and corresponding detail view. The device consists of a filtration media, in this case, a membrane 100, which in an embodiment is an asymmetric polysulfone filter, but can include other permeable or semi-permeable polymers which would selectively allow for passage of blood plasma without transmission of cellular content. The filtration media is used to separate two fluid chambers 101 and 102. The walls of the chamber are a polycarbonate material in an embodiment, but can also be made of other biocompatible polymers or metals, including ultra-high molecular weight polyethylene, stainless steel, or titanium. The filtration media is supported to prevent displacement by two filter support grates 103 and 104, composed of a biocompatible metal or polymer, stainless steel in an embodiment. Flow across the filter is induced and controlled in this embodiment by two separate pistons, 105 and 106, coupled to independent drive mechanisms 107 and 108, in this case, shown as threaded rods, but could alternatively include other mechanisms such as pneumatic or hydraulic cylinders, or any other means of providing rate controlled displacement of the piston. In an embodiment, the pistons are made of biocompatible stainless steel, but can also be fashioned from other biocompatible metals, such as titanium, or polymers, such as ultra-high molecular weight polyethylene. The drive rods in an embodiment are steel, but can include other metals such as aluminum, brass, or any other material non-similar to the interface on the piston. Further depicted in the figure on the piston face are a series of recesses 109 and 110, intended to allow for the piston to be seated against the filter 100 without being obstructed by the filter support grates 103 and 104. Features 111 allow for alignment of the filter support grates and the fluid chambers. A seal 112, shown in this embodiment as an O-ring, is intended to prevent fluid escape from the chamber. Piston O-rings 113 and 114 are utilized to prevent fluid and gas escape as the pistons 105 and 106 are displaced. The seals allow for changes in pressure which induce the flow in the chambers. All O-rings are silicone in an embodiment, but could be substituted by an appropriate biocompatible elastomer. A port 115 is provided to allow fluid to enter and escape from the intake chamber of the device from and to external reservoirs not depicted in the figures.

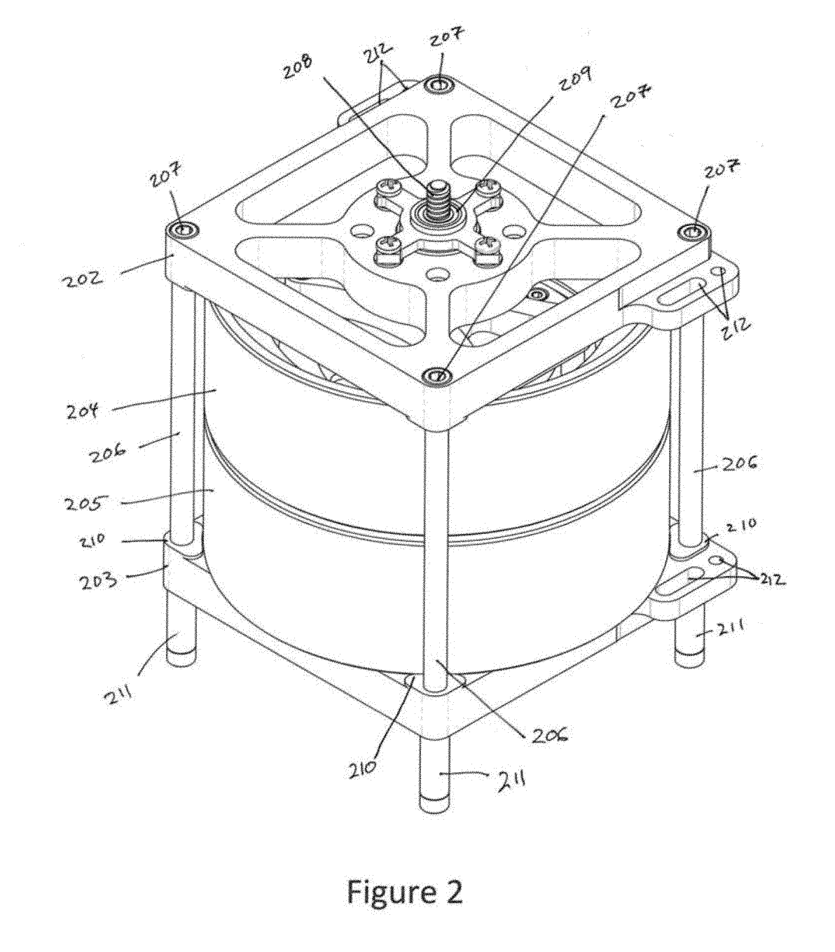

FIG. 2 depicts a non-section view of the embodiment depicted previously in FIG. 1. Shown in the figure is a framework which consists of endplates 202 and 203 intended to interface against the filter chambers 204 and 205 and provide compression on the chambers for assembly and compression of the O-ring shown previously as 112. The compression is induced by members 206 which are coupled to the endplates, in this embodiment by threaded fasteners 207, but which could be replaced by another fastening mechanism, such as a cross-pin, rivet, or other device. The endplates additionally provide alignment for the drive mechanisms, of which only one, 208, is seen. In the threaded drive embodiment shown, roller bearings, seen as 209, are utilized to allow for support of the drive mechanism without creating increased friction and hampering function. Further in this embodiment are features 210 which provide locational alignment for the filter chambers and further prevent misalignment during device assembly. Additional features such as legs 211 are utilized to maintain clearance of the drive mechanism, but may not be required in other embodiments. Features 212, depicted in this figure as slots and threaded holes may be utilized for assembly to other components, such as other filter chamberings when used in parallel, or a drive mechanism as depicted in further embodiments.

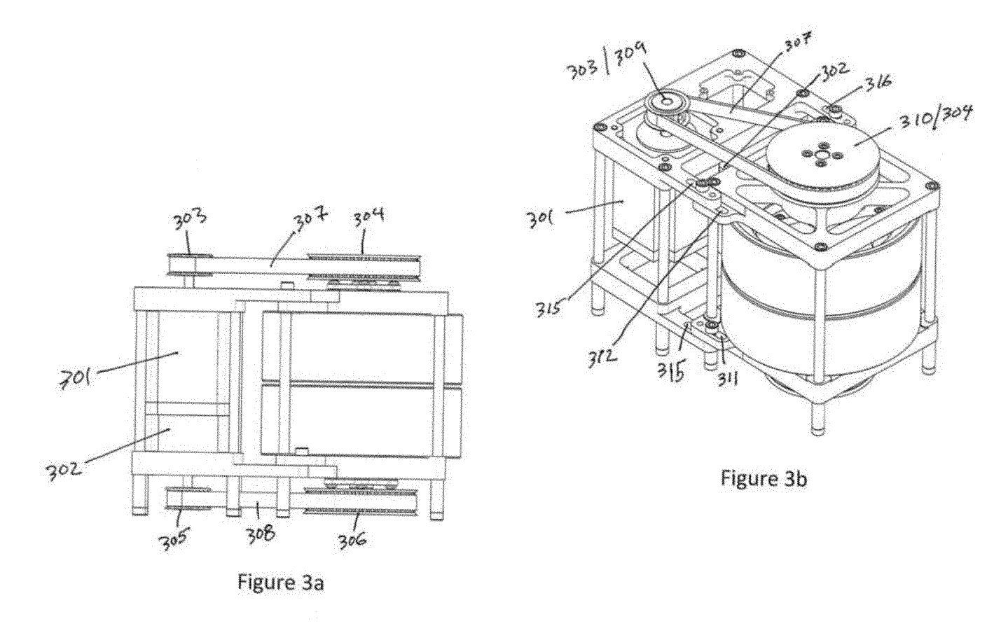

FIG. 3a depicts an embodiment of the device in which the piston displacement is induced by one or more stepper motors, 301 and 302 in this particular form. The embodiment further depicts a set of drive pulleys 303, 304, 305, and 306. Seen better in FIG. 3b, timing belts 307 and 308 are used to couple the drive pulleys, transferring rotational motion from the stepper motors to the threaded drive, and subsequently inducing linear motion of the piston. The combination of the drive pulleys, such as the combination of pulleys 309 and 310 in FIG. 3b, can be tailored in size to gear the rotational motion. This can be used to increase the range of speed or force that can be applied using the motors. As previously described in FIG. 2, features, such as 311, 312, on the device can be used to couple the filter device to the motors via corresponding features on the drive device, seen here as 315. While this particular embodiment depicts a series of stepper motors, drive pulleys, and timing belts, other embodiments induce rotary motion through other motor types, such as servo motors, other driver features, such as gears, sprockets and chains, or direct coupling of the motor to the threaded drive shaft. Manual application of the rotary motion can also be applied with a crank or hand wheel. These embodiments are in addition to other methods of initiating the motion such as the pneumatic and hydraulic cylinders intimated previously. Not depicted in the figure is a microcontroller which provides the signaling for speed and volume of fluid flow to be moved. Other embodiments may include manual application of force through the use of a hand crank, coupled with a spring to store the energy to be applied to the system, and may not use a microcontroller.

FIGS. 4a and 4b depict the initial state of the device prior to function in detailed and simplified forms, respectively. In each case, the pistons 401 and 402 are brought into contact or near contact with the filter medium 403. The filter support grates 404 and 405 are in close contact with the recesses in the piston face for each piston. In this state, dead space, such as 406, between the filter support grate and piston face is minimized and may be filled with air, or a specified gas and or fluid combination prior to usage. The entry and/or exit port(s), shown as a single port 407 in this embodiment, are also filled with air, specified gas or fluid at this state.

FIGS. 5a and 5b depict the intake state of the device, when the initial cell suspension is drawn into the device, in detailed and simplified forms, respectively. In each case, intake piston 501 is displaced in a direction away from the filter medium 502. Because the filter chambers are sealed, a vacuum is created in the intake chamber 503. This vacuum induces flow of the unconcentrated cell suspension 504 through intake port 505 or ports. This allows for uniform distribution of the cell suspension across the face of the intake piston 501 and subsequently, the filter medium 502. As the filter medium is selectively permeable, it does not, in an embodiment, allow passage of air after wetting. By drawing the solution in from a location distal to the filter medium, the cell suspension may avoid contact with the filter medium until the chamber is filled and any trapped air or gas has moved through the filter medium to the top of the dead-end chamber 506 (and doing so in an automated manner that does not leave the flow rate to the discretion of the user, whereby the user may use too much force to inject the fluid and consequently spray the fluid on the membrane prematurely before the majority of gases have been pushed through the filter). The flow rate into the chamber can be controlled by controlling the speed of the piston travel. In an embodiment, the piston can vary in speed to accommodate the difference in cross section area of the intake chamber when the piston is engaged in the filter support grate 507 versus when the piston has cleared the filter support grate, as shown in the respective figures. Additionally, in an embodiment the flow rate of the piston varies to vary the flow rate as a function of distance from the filter support grate, either to control the dispersion of the solution or to reduce burden on the drive mechanism. In an embodiment the un-concentrated solution enters the chamber at a rate between 0.25 ml/s and 5 ml/s. Such a flow rate may be programmed into a controller-based system described below.

FIGS. 6a and 6b depict the filtration state of the device, when the fluid component of the initial cell suspension has been forced through the filter, in detailed and simplified forms, respectively. In each case, intake piston 601 is displaced in a direction toward the filter medium 602 while the dead-end piston 603 is moved in a direction away from the filter medium. As the filter chambers are sealed, a vacuum is created in the dead-end chamber 604, while the volume of the intake chamber 605 is reduced to prevent additional fluid from entering the chambers. This motion induces flow of the fluid component of the unconcentrated cell suspension 606 through the filter. The cellular content is trapped in the spaces between the intake piston and the filter support grate 607 on the intake side, or on the surface of the filter medium 608. In the described embodiment, the filter medium allows fluid, but no cellular content to pass. The medium may be changed to allow for platelets or other smaller cells to pass through the membrane to allow for different end products. The flow rate through the filter and into the dead-end chamber can be controlled by controlling the speed of each piston's travel. The piston can vary in speed to accommodate the difference in cross section area of the intake chamber when each piston is engaged in its relative filter support grate 609 and 610 versus when the piston has cleared the filter support grate. Additionally the flow rate of the piston is variable to allow the flow rate to vary as a function of distance from the filter support grate, either to control the dispersion of the solution or to reduce burden on the drive mechanism. In this embodiment, the un-concentrated solution flows through the filter medium at a rate between 0.1 ml/s and 2 ml/s. Such a flow rate may be programmed into a controller-based system described below.

FIGS. 7a and 7b depict the backflush state of the device, when the fluid component of the initial cell suspension is forced through the filter in a reverse direction, in detailed and simplified forms, respectively. In each case, intake piston 701 is displaced in a direction away from the filter medium 702, while the dead-end piston 703 is moved in a direction toward the filter medium. The magnitude of the movement in this step is a fraction (half or less) of the movement of the filtration step (FIGS. 6a and 6b). As the filter chambers are sealed, pressure is created in the dead-end chamber 704, while the volume of the intake chamber 705 is increased to create a vacuum and space for the fluid to flow. By maintaining the total overall volume in the system, no fluid enters or leaves the system. This motion induces flow of the fluid component 706 backward through the filter. The cellular content which was previously trapped and deposited on the surface of the filter 707 is forced back into the suspension 708 on the intake side. As a smaller volume of the fluid component is moved into the intake chamber, with the same cellular content (as the filter prevented the cells from flowing into the dead-end chamber, the resulting concentration of the cell suspension is greater than the original un-concentrated cell suspension). The flow rate through the filter can be controlled by controlling the speed of each piston's travel. The piston can vary in speed to accommodate for the difference in cross section area of the intake chamber when each piston is engaged in its relative filter support grate 709 and 710 versus when the piston has cleared the filter support grate. Additionally, the travel rate of the piston could also vary to vary the flow rate as a function of distance from the filter support grate, either to control the dispersion of the solution or to reduce burden on the drive mechanism. In an embodiment, the un-concentrated solution flows through the filter medium at a flow rate between 10 ml/s and 50 ml/s. Notably in an embodiment this flow rate is faster than say, for example, the flow rate for FIG. 6(b) (0.1 ml/s and 2 ml/s). This allows a movement closer to a “pulse” to eject cells forcibly from the membrane without having to pass excess fluid from the upper chamber to the lower chamber.

FIGS. 8a and 8b depict the ejection state of the device, when the concentrated cell suspension is forced out of the device, in detailed and simplified forms, respectively. In each case, intake piston 801 is displaced in a direction toward the filter medium 802, while the dead-end piston 803 remains static. As the filter chambers are sealed, no fluid can flow out of the dead-end filter chamber 804. The increase in pressure in the intake chamber 805 forces fluid out of the output port 806 and the concentrated cell suspension 807 is allowed to flow through said port. In one embodiment, that flow is allowed by a check valve with a cracking pressure that is not reached during the filtration process, but is reached during the ejection phase. In an alternate embodiment, the check valves are replaced with a valve controlled with the microcontroller. In one embodiment, the valves are solenoid driven pinch valves. In other embodiments, these may be rotary stopcock valves powered by a motor, or any other type of valve which may be actuated using an electronic signal or a pneumatic, hydraulic, or manually applied force. The flow rate from the device can be controlled by controlling the speed of the intake piston's travel. The piston can vary in speed to accommodate for the difference in cross section area of the intake chamber when each piston is engaged in its relative filter support grate versus when the piston has cleared the filter support grate. Additionally, the travel rate of the piston could also vary to vary the flow rate as a function of distance from the filter support grate, either to control the dispersion of the solution or to reduce burden on the drive mechanism. In an embodiment the un-concentrated solution flows through the filter medium at a flow rate between 0.25 ml/s and 5 ml/s. Such a flow rate may be programmed into a controller-based system described below.

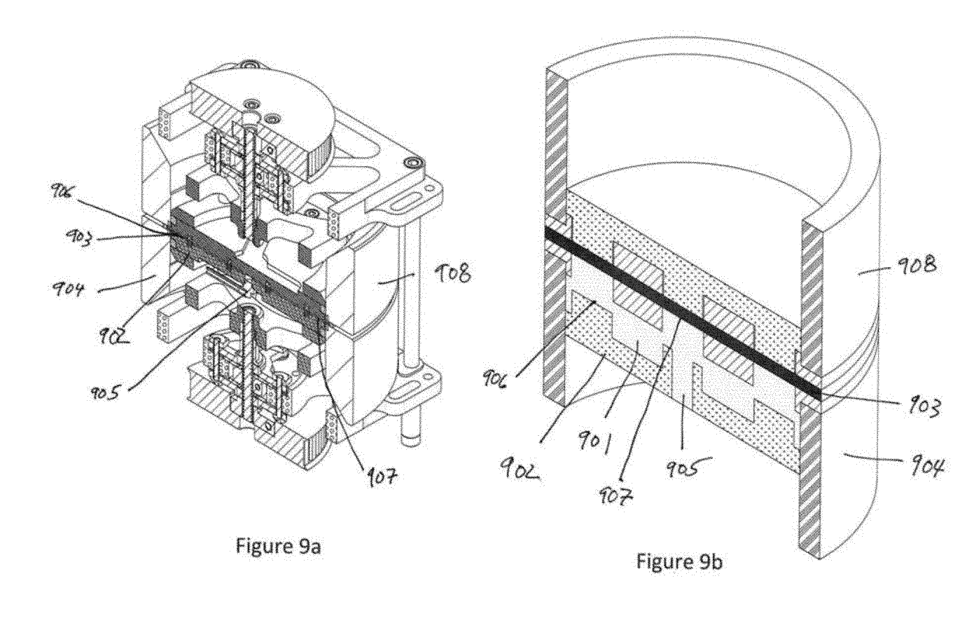

FIGS. 4a through 8b provide a method and system for filtering cell suspensions. However, other methods of achieving that function with the same embodiment are anticipated. FIGS. 9a through 11b provide an alternate intake method which utilizes a small buffer portion of the unconcentrated cell suspension to allow for space to be created for flow without pulling the entirety of the intake volume into the intake chamber. This can serve the purpose of reducing the total amount of time needed to complete the process as the entire volume does not have to first be drawn into the device and then filtered. Instead, the small buffer portion is drawn into the device, and then the remaining volume is drawn directly through the filter and into the dead-end chamber. The subsequent descriptions of FIGS. 9a-11b further describe the steps in this alternative method.

FIGS. 9a and 9b depict an optional buffer state of the device, in detailed and simplified forms, respectively. This embodiment utilizes the same mechanism (i.e., system, such as system of FIG. 1) for completion of the end goal, but utilizes a different method. For this use case, a small buffer portion of the unconcentrated cell solution 901 is drawn into the device in the same manner as the previously described intake method, but at a smaller volume (10 to 50% of the overall volume to be filtered). In each case, intake piston 902 is displaced in a direction away from the filter medium 903. As the filter chambers are sealed, a vacuum is created in the intake chamber 904. This vacuum induces flow of the unconcentrated cell suspension through an intake port 905 or ports. This allows for uniform distribution of the cell suspension across the face of the intake piston 906 and subsequently, the filter medium 907. As the filter medium is selectively permeable, it may or may not allow passage of air after wetting. By drawing the solution in from a location distal (below) to the filter medium, the cell suspension may avoid contact with the filter medium until the chamber is filled (and therefore any trapped air or gas may move to the top of the dead-end chamber 908). A goal of the distal location is to avoid wetting (fluid contact) of the filter as it becomes impermeable to gas once wetted in some embodiments.

The flow rate into the chamber can be controlled by controlling the speed of the piston travel. The piston can vary in speed to accommodate the difference in cross section area of the intake chamber when the piston is engaged in the filter support grate 909 versus when the piston has cleared the filter support grate, as shown in the respective figures. Additionally, the travel rate of the piston can also vary to vary the flow rate as a function of distance from the filter support grate, either to control the dispersion of the solution or to reduce burden on the drive mechanism. In this embodiment, the un-concentrated solution enters the chamber at a rate between 0.25 ml/s and 5 ml/s. Such a flow rate may be programmed into a controller-based system described below.

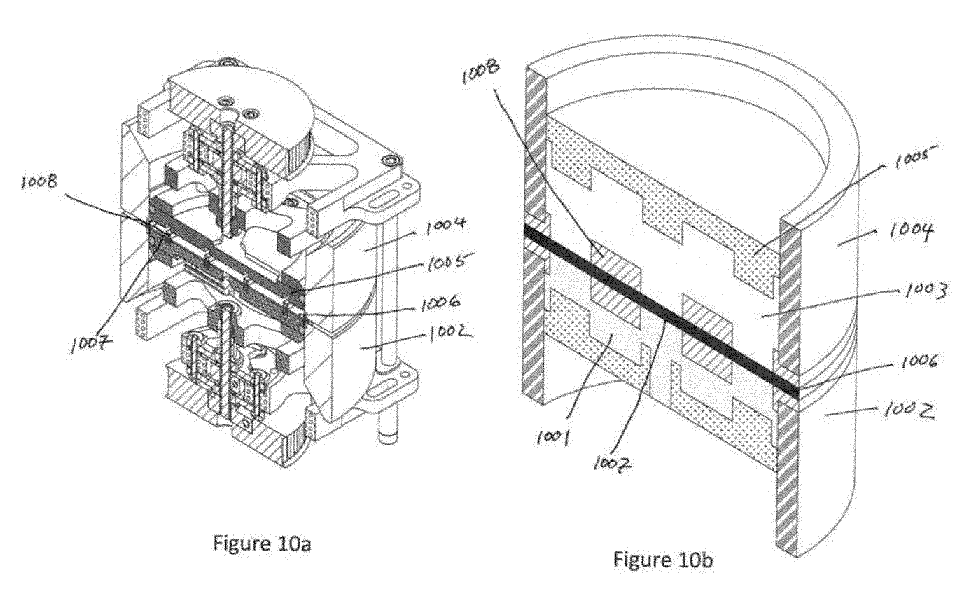

FIGS. 10a and 10b depict an alternative filtration state of the device, in detailed and simplified forms, respectively. For this use case, the unconcentrated suspension 1001 is drawn into the intake chamber 1002 and the fluid component 1003 of the unconcentrated cell solution is drawn through the filter medium 1006 and into the dead-end chamber 1004 at the same time. The dead-end piston 1005 is displaced in a direction away from the filter medium 1006. As the filter chambers are sealed, a vacuum is created in the chambers. This vacuum induces flow of the unconcentrated cell suspension through an intake port 1007 or ports and the fluid component then flows through the filter media into the dead-end chamber, with the cells being trapped in the intake chamber by the filter. The previously created buffer layer allows for uniform distribution of the cell suspension across the face of the filter 1007. In other words, the buffer method may allow for better cell distribution across the membrane (as compared to the first method). In the first method the entirety of the fluid to be concentrated is introduced into the system while piston 501 is far away from the membrane (transition from FIG. 4B to FIG. 5B). The fluid is drawn into the system in one phase. But with the buffer layer method the fluid is drawn into the system in two or more phases. A first small phase occurs (FIG. 9B) and then a second phase occurs where fluid is introduced into fluid from the first phase (i.e., note how lower piston does not move down when transitioning from FIG. 10a to FIG. 10B). As the second phase fluid moves into the first phase fluid it is easier for the cells to evenly distribute than would be the case if that buffer fluid (the phase 1 fluid) were not already there (e.g., it is easier for cells to distribute within fluid rather than within an air). Further, without a buffer layer and with the intake piston not displaced from the filter medium any unconcentrated cell solution would be drawn through the port in the intake piston and flow directly into the dead-end chamber. The result is that all fluid flow into the dead end chamber occurs in the immediate location around the intake port. The result is that air may be trapped in the intake chamber as the filter is wetted on the dead end chamber side. Additionally, as the flow into the dead end chamber is focused nearest the intake port, Applicant determined the filter may become overloaded with cells in that area, reducing filtration efficiency and potentially leading to rupture of the filter. The buffer volume allows for fluid to disperse in the intake chamber before flowing into the dead end chamber, resulting in similar results as the previously described method. The benefit of this methodology is that a smaller volume is drawn into the intake chamber, allowing for time savings. The flow rate into the chamber can be controlled by controlling the speed of the piston travel. The piston could vary in speed to accommodate the difference in cross section area of the chamber when the piston is engaged in the filter support grate 1008 versus when the piston has cleared the filter support grate, as shown in the respective figures. Additionally, the travel rate of the piston could also vary to vary the flow rate as a function of distance from the filter support grate, either to control the dispersion of the solution or to reduce burden on the drive mechanism. In an embodiment, the fluid component is filtered into the dead-end chamber at a rate between 0.1 ml/s and 2 ml/s. Such a flow rate may be programmed into a controller-based system described below.

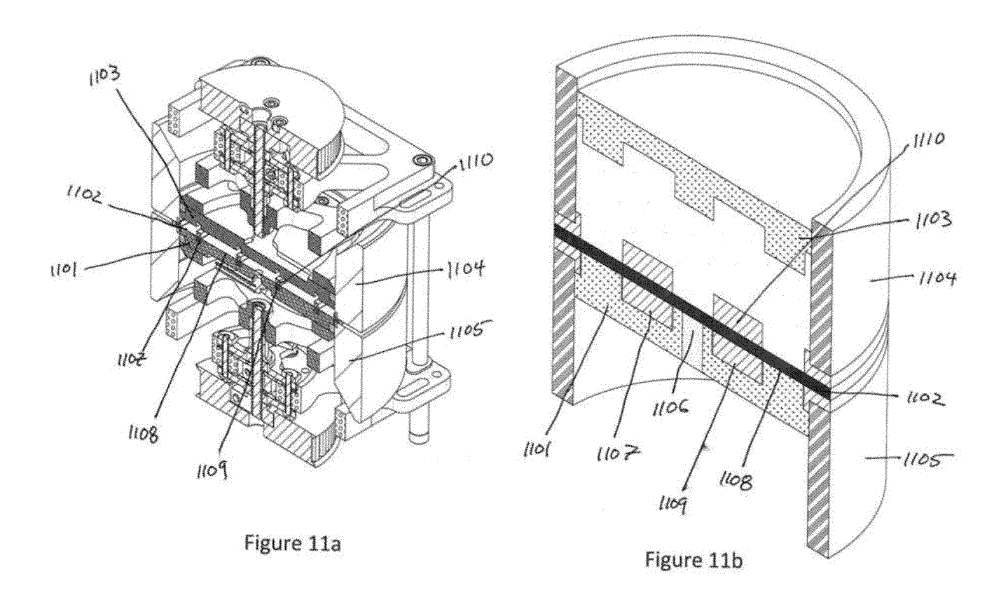

FIGS. 11a and 11b depict the de-buffer state of the device, when the fluid component of the unconcentrated cell suspension buffer is forced through the filter, in detailed simplified forms, respectively. In each case, intake piston 1101 is displaced in a direction toward the filter medium 1102 while the dead-end piston 1103 is moved in a direction away from the filter medium. As the filter chambers are sealed, a vacuum is created in the dead-end chamber 1104, while the volume of the intake chamber 1105 is reduced to prevent additional fluid from entering the chambers. This motion induces flow of the fluid component of the remaining unconcentrated cell suspension 1106 through the filter. The cellular content is trapped in the spaces between the intake piston and the filter support grate 1107 on the intake side, or on the surface of the filter medium 1108. The flow rate through the filter and into the dead-end chamber can be controlled by controlling the speed of each piston's travel. The piston may vary in speed to accommodate the difference in cross section area of the intake chamber when each piston is engaged in its relative filter support grate 1109 and 1110 versus when the piston has cleared the filter support grate. Matching the volumetric flow rate prevents a change in pressure of the chambers as a whole, thus not drawing in additional fluid, or forcing fluid out, by elevating the pressure difference above the cracking pressure of the one way check valve. Additionally, the travel rate of the piston could also vary to vary the flow rate as a function of distance from the filter support grate, either to control the dispersion of the solution or to reduce burden on the drive mechanism. In an embodiment, the un-concentrated solution flows through the filter medium at a rate between 0.1 ml/s and 2 ml/s.

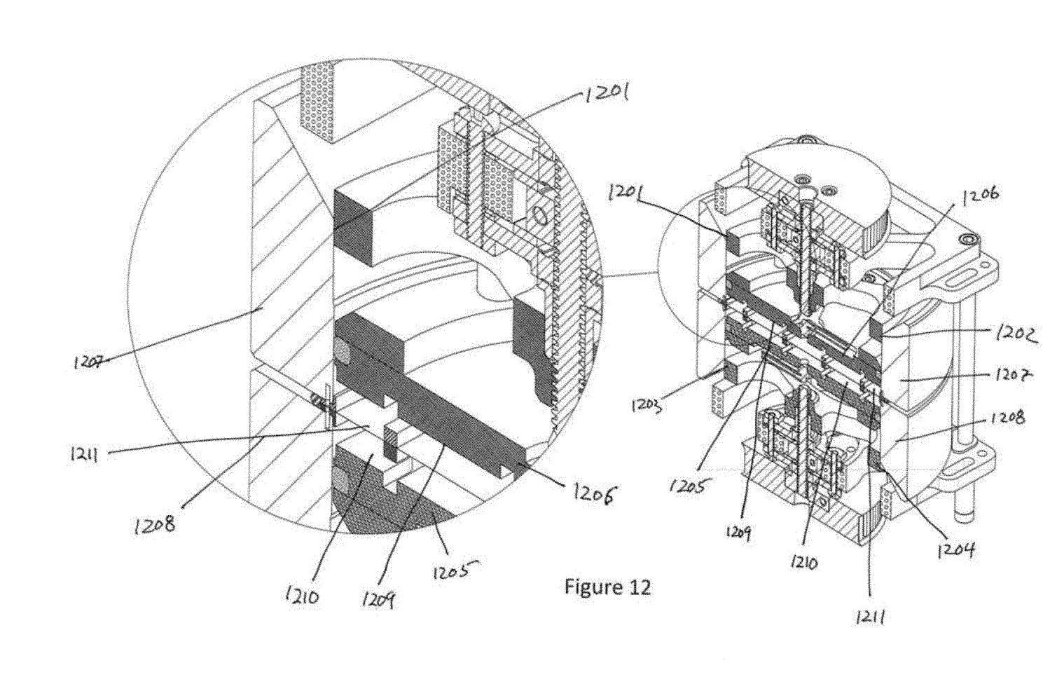

FIG. 12 depicts an embodiment of the invention in which additional bearing surfaces 1201, 1202, 1203, and 1204 are included between the pistons 1205 and 1206 and the chamber walls 1207 and 1208. The bearing surfaces, being an integral part of the piston assembly, move with the piston and allow for additional contact at locations between the face of each piston 1209 and 1210 and the outer end of the device with the intent of reducing the overall amount of tilt that can occur in the piston, to keep the piston faces parallel to the filter medium 1211. Such features ensure the seal remains consistent around the edge of the pistons to prevent loss of seal integrity. In other embodiments of the device, features to maintain the rotational orientation of the piston are also anticipated in various forms such as guide rods, key ways, etc.

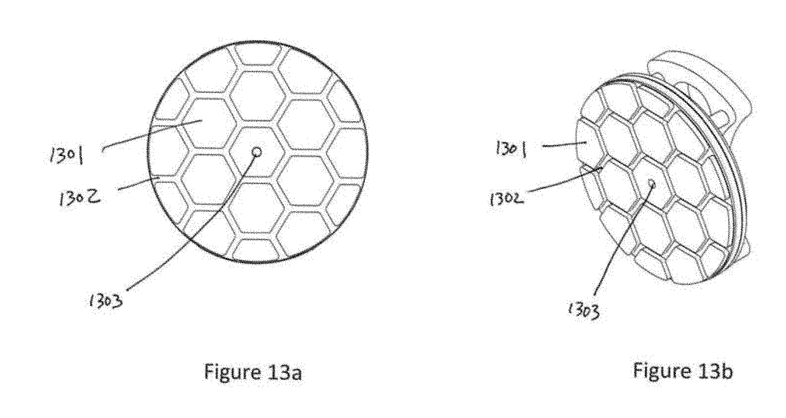

FIGS. 13a and 13b depict a view of the piston face and an isometric view of the piston, respectively. As discussed prior, the piston face contains a number of protrusions 1301, formed by a number of recesses 1302 intended to interface with the filter support grate. This particular embodiment demonstrates a pattern of hexagonal-shaped protrusions in a “honeycomb” pattern. In other embodiments the shape and pattern of the protrusions are changed to provide optimal exposure of the filter medium while maintaining adequate support. Additionally, the figures depict a port 1303 which is configured to attach to a hollow cannula for transportation of fluid into and out of the filter chamber. The cannula contains a number of one-way flow valves that control the flow of fluid from an intake reservoir and into an output reservoir. For the embodiments described in FIGS. 1 through 12, the ports are only required on the intake chamber. In additional embodiments, ports are included on the dead-end chamber piston for various functions, including monitoring of pressure and allowing removal of filtered plasma.

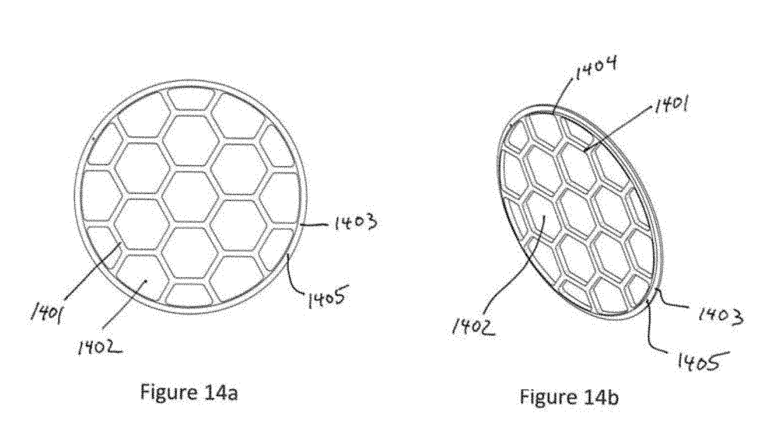

FIGS. 14a and 14b depict the face of and an isometric view of a filter support grate, respectively. The filter support grate allows for support of the filter to prevent bowing or sagging of the filter. Sagging or bowing can induce contact which could impede the flow through the out port(s) of the piston. The sagging or bowing also results in a volume change in each chamber, resulting in unpredictable yields. In each case, a number of connected members 1401 connect to create a number of voids 1402 in the center of the grate. The interconnected members are intended to mate to the recesses of the pistons (shown previously as 1302), while the previously shown piston protrusions (1301) mate to the filter grate voids. Additionally, a flange 1403 is created on the filter support grate which prevents it from moving into the filter chamber during the filtration process. The outer surface 1404 of the main grate mates to the inner wall of the chamber to maintain its position. Additionally, features to maintain alignment, such as the pin holes 1405 may be incorporated into the grate. In other embodiments, the filter support grate is integrated into the filter and does not extend beyond the surfaces of the filter. In those embodiments, the piston could be flat, as there is no need for recessed channels to allow complete collapse of the chamber.

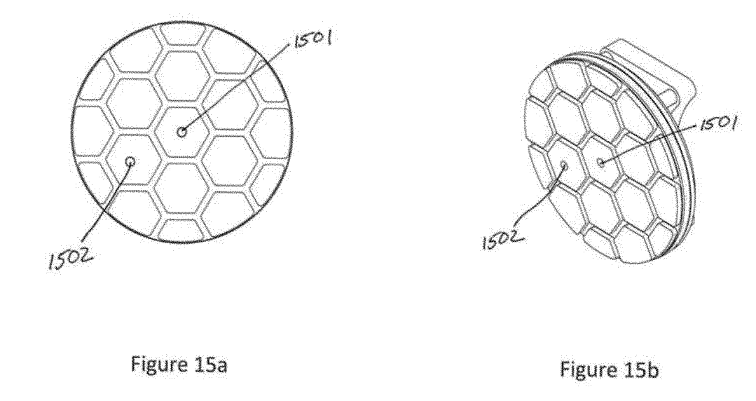

FIGS. 15a and 15b depict a view of another embodiment of the piston face and an isometric view of the piston, respectively. Specifically, this figure demonstrates an embodiment in which two ports 1501 and 1502 are present. In this embodiment, each may be configured to attach to a hollow cannula for transportation of fluid. In this embodiment, one port, 1502, for example, could be utilized for moving fluid into the filter chamber, while the other port, 1501, for example, could be used for moving fluid out of the chamber. In this embodiment, each cannula contains a one-way flow valve that controls the flow of fluid from an intake reservoir and into an output reservoir. Both ports can also be used for moving fluid into and out the chamber by uniting the connected cannulas on each port to a manifold system. For the embodiments described in FIGS. 1 through 12, the ports are only required on the intake chamber. In additional embodiments, ports could be included on the dead-end chamber piston for various functions, including monitoring of pressure and allowing removal of filtered plasma.

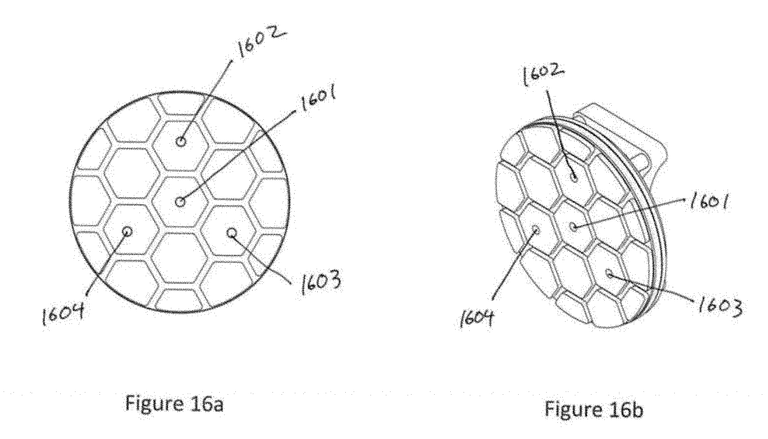

FIGS. 16a and 16b depict a view of another embodiment of the piston face and an isometric view of the piston, respectively. Specifically, this figure demonstrates that a plurality of ports is also anticipated. This embodiment contains multiple ports, 1601, 1602, 1603, and 1604. In this embodiment, each may be configured to attach to a hollow cannula for transportation of fluid. In this embodiment, one port, 1601, for example, could be utilized for moving fluid into the filter chamber, while the other ports, 1602, 1603, and 1604, for example, could be used for moving fluid out of the chamber. In this embodiment, each cannula contains a one-way flow valve that controls the flow of fluid from an intake reservoir and into an output reservoir. Other combinations of input and output ports can be used (two input ports and two output ports, as a non-exclusive example). Any of the ports can also be used for moving fluid into and out the chamber by uniting the connected cannulas on each port to a manifold system. For the embodiments described in FIGS. 1 through 12, the ports are only required on the intake chamber. In additional embodiments, ports could be included on the dead-end chamber piston for various functions, including monitoring of pressure and allowing removal of filtered plasma.

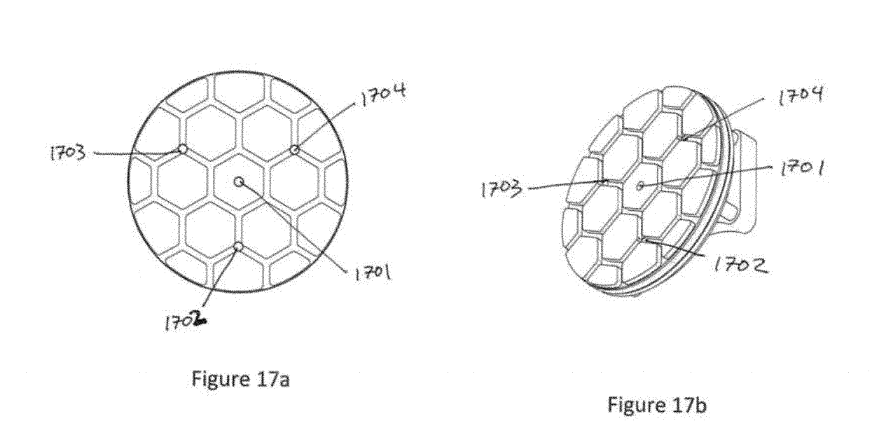

FIGS. 17a and 17b depict a view of another embodiment of the piston face and an isometric view of the piston, respectively. Specifically, this figure demonstrates that a plurality of ports is also anticipated and that the location of the ports can occur in the recesses of the piston face in addition to the protrusions of the piston face. Applicant determined that this embodiment allows for the case of fast cell settling, where the cells fall into the recesses in the piston face. Applicant determined by locating the ports in the recesses, more cells are harvested during the ejection phase, without potentially being trapped by the filter support grate. This embodiment contains multiple ports, 1701, 1702, 1703, and 1704. In this embodiment, each may be configured to attach to a hollow cannula for transportation of fluid. In this embodiment, one port, 1701, for example, could be utilized for moving fluid into the filter chamber, while the other ports, 1702, 1703, and 1704, for example, could be used for moving fluid out of the chamber. In this embodiment, each cannula contains a one-way flow valve that controls the flow of fluid from an intake reservoir and into an output reservoir. Other combinations of input and output ports can be used (two input ports and two output ports, as a non-exclusive example). Any of the ports can also be used for moving fluid into and out the chamber by uniting the connected cannulas on each port to a manifold system.

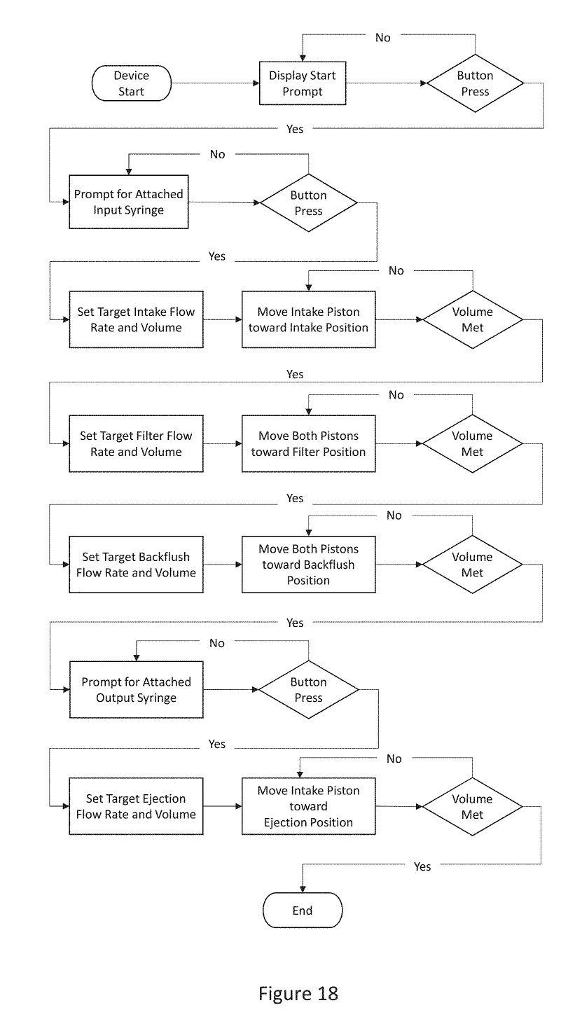

FIG. 18 provides a method of operation utilizing a device such as the embodiments depicted in the prior figures. Briefly, the device starts and displays a prompt to the user. After a user activation, shown in the figure as a button press but in other embodiments by a mechanical switch, the system prompts for the input syringe to be attached. In one embodiment, this portion is replaced by a mechanical switch that is activated with the attachment of a syringe. In yet other embodiments, this can be replaced by an optical sensor or another method for detecting the presence of the input syringe. After the attachment of the input syringe or other fluid containment vessel, the filtration and concentration process begins.

The process may be automated by a controller in cooperation with at least one motor, such as a step motor. A stepper motor or step motor or stepping motor may be a brushless DC electric motor that divides a full rotation into a number of equal steps. The motor's position can then be commanded to move and hold at one of these steps without any feedback sensor (an open-loop controller). While stepper motors are used in some embodiments, in other embodiments flow rate and volume that has flowed may be assessed with sensors such as infrared sensors, phototransistors, and the like.

The input flow rate and target volume is set in the software, after which the input piston is moved to start the inflow of fluid into the intake chamber. At each step, a loop completes (not shown in flow chart) during which the software checks to determine if it is time for a step to be taken per the input flow rate. If it is time, a step is taken. After each step, the software checks to determine if the desired volume of fluid has been moved. In one embodiment of the invention, the volume is calculated based on the number of steps taken and calculation factors for conversion of the number of pump steps into volume of fluid. In another embodiment, this is done with a sensor that monitors the position of the piston. If not, the motor is again stepped (using the same logic loop to check maintain the flow rate). If the fluid level has been met, the software proceeds to the next state. The next step starts with setting the filtration flow rate and volume. During filtration, both pistons are moved using the same loop structure for maintaining the piston speed and managing the volume of fluid movement. Upon exit of the filtration move loop, the backflush flow rate and volume are set. A move loop is then used to gain maintain the piston speed and volume of fluid moved. After backflush, a safeguard is included to ensure that an output syringe (or other fluid vessel) is attached. In the figure, this is achieved by a confirmation button press when the syringe is attached. As with the intake syringe confirmation, in other embodiments, this may be a mechanical switch. In another embodiment, this portion is replaced by a mechanical switch that is activated with the attachment of a syringe. In yet other embodiments, this can be replaced by an optical sensor or another method for detecting the presence of the output syringe. At various times the movement of pistons is simultaneous (i.e. the piston may not necessarily start and/or stop movement at the same time but they are both moving at one point in time). This occurs at, for example, between FIGS. 6(B) and 7(B) when one piston is engaged with the filter support grate and the other is not, and so must move at different rates of speed to achieve a constant volume across the combined volume of the two chambers. At other times, only one piston is moving such as, for example, the transition between FIG. 4(B) to FIG. 5(B).

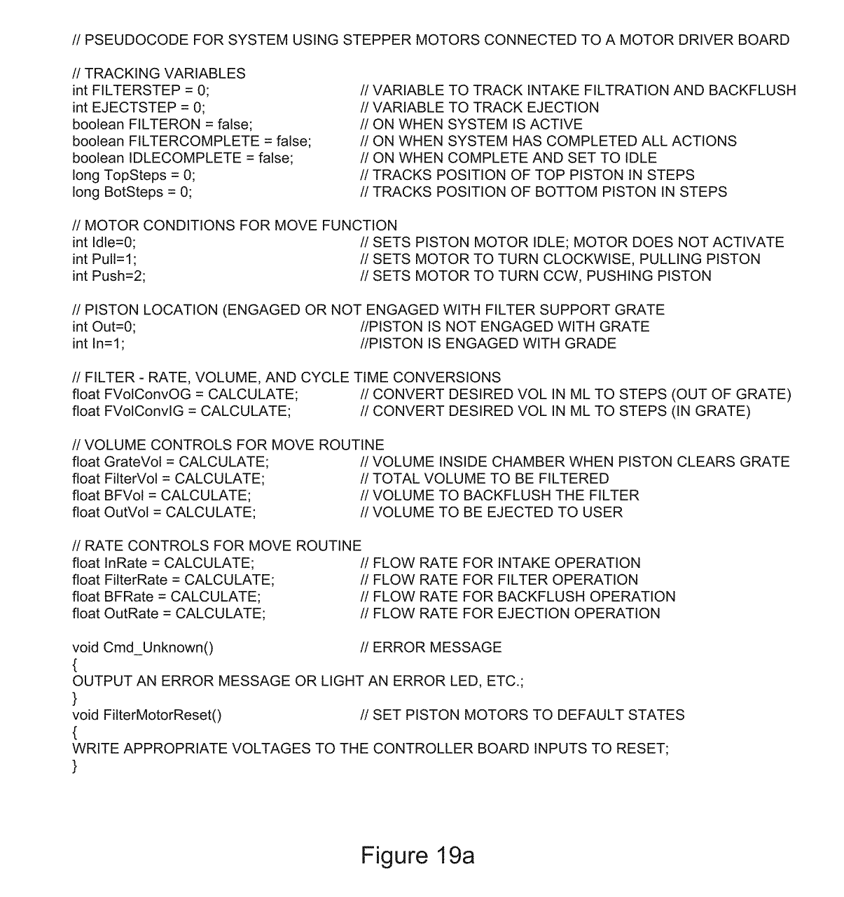

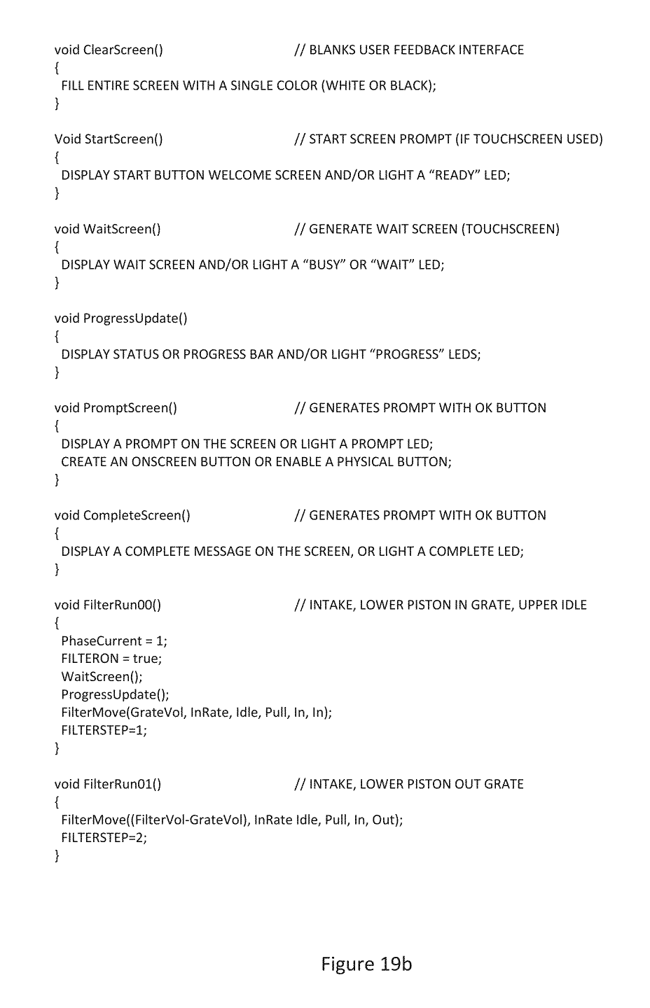

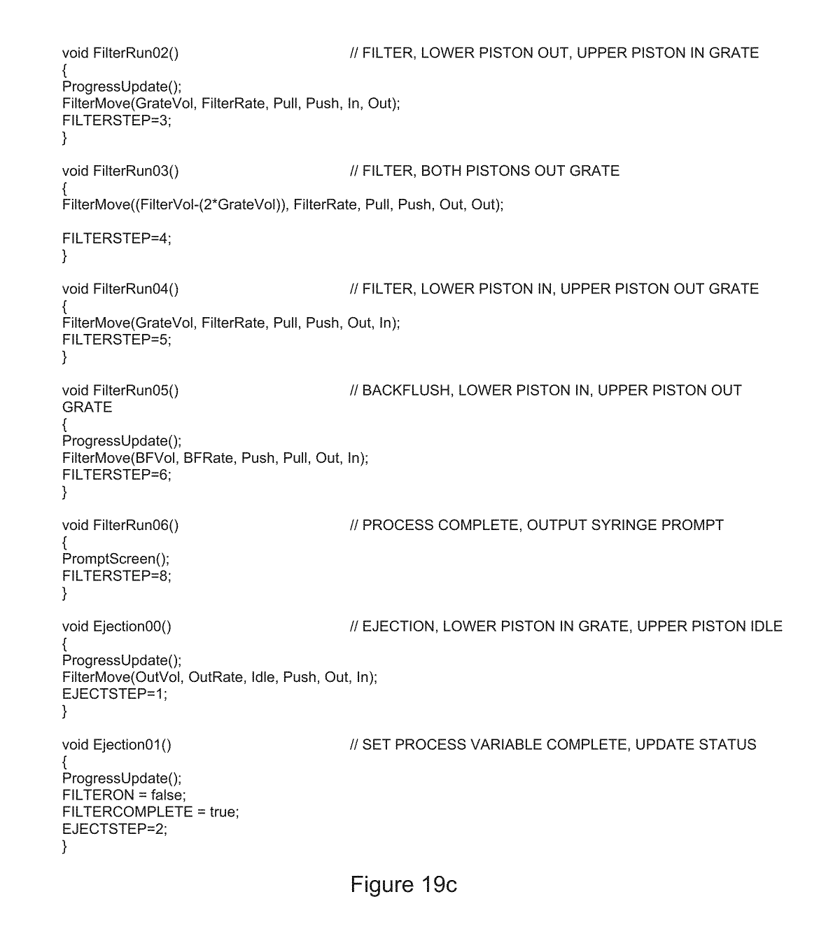

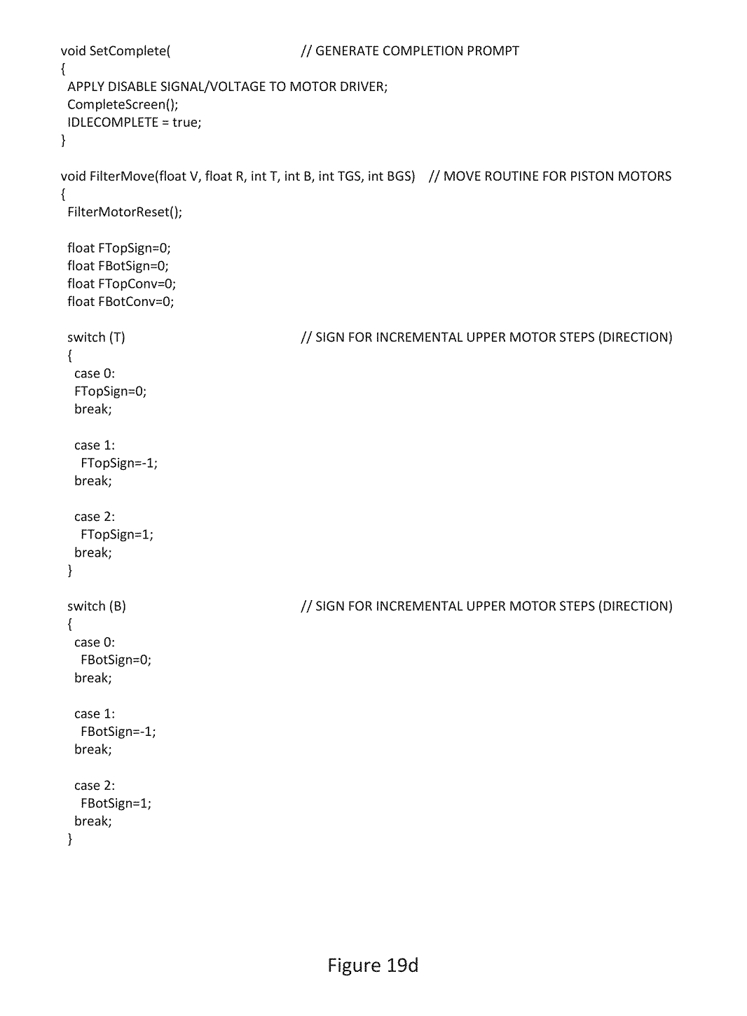

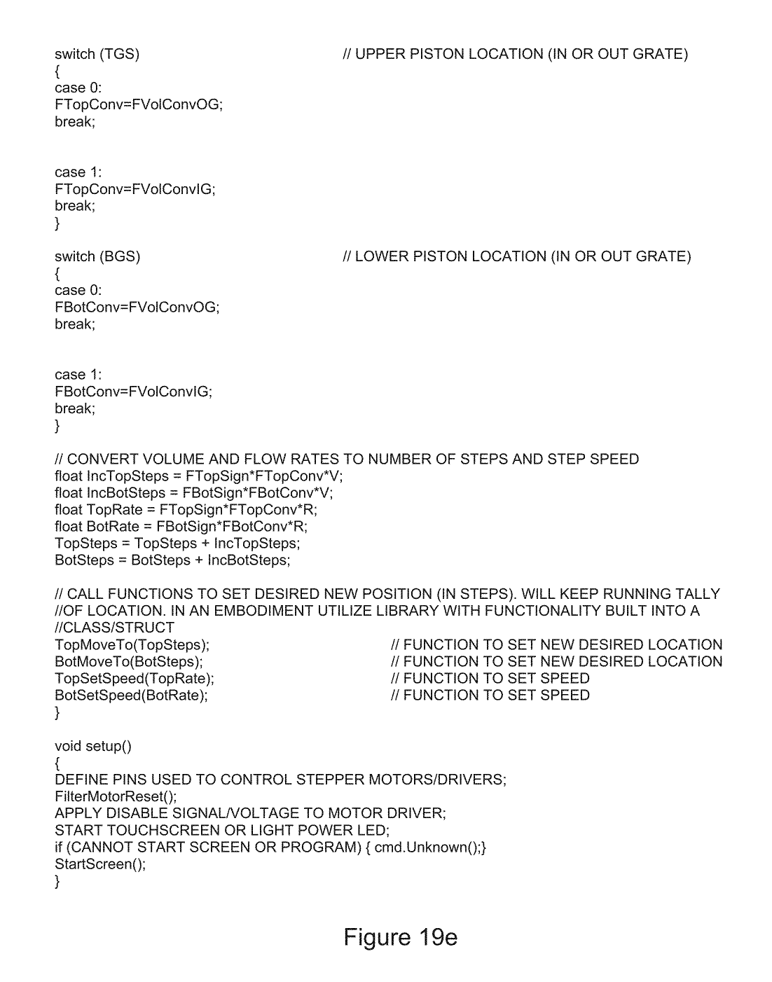

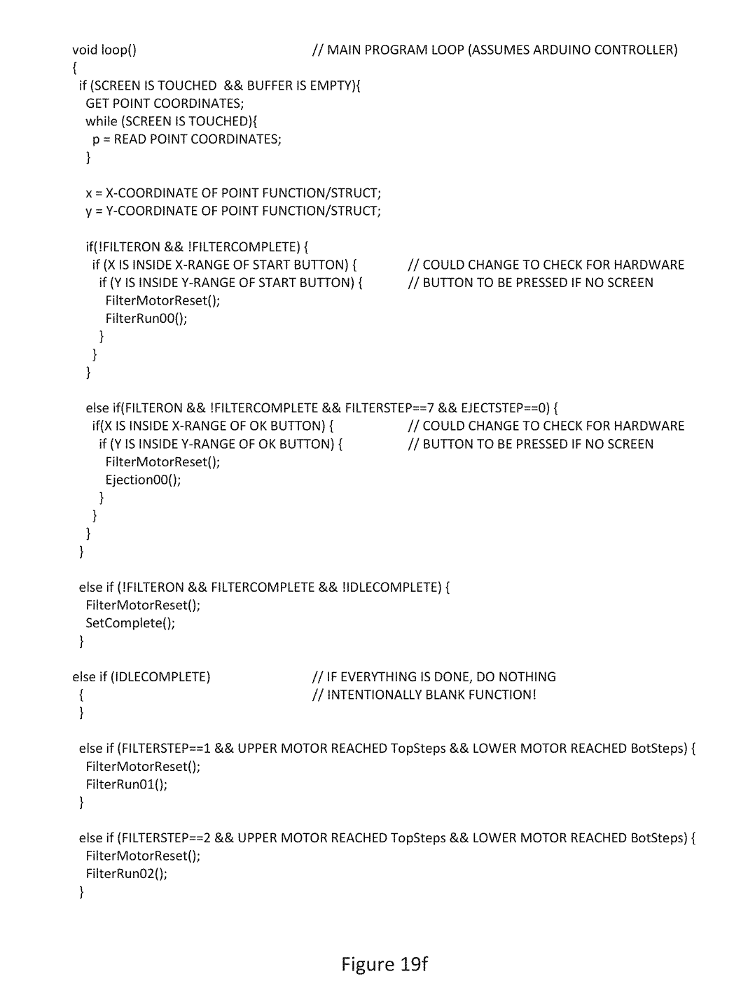

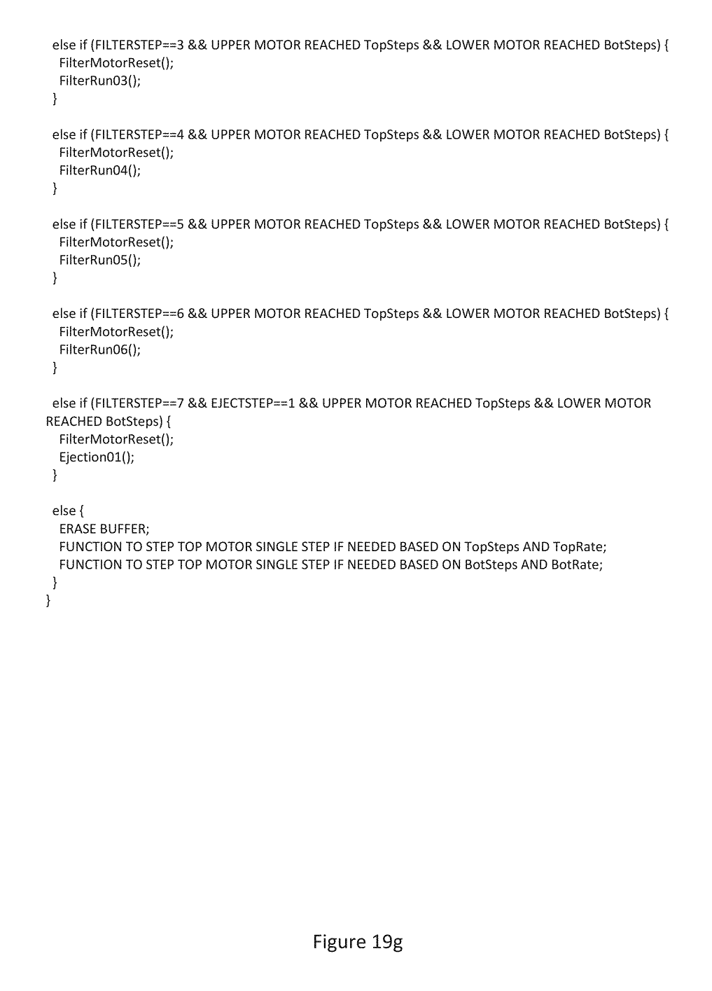

FIGS. 19a through 19g depict pseudocode for a microprocessor which controls the signals sent to the controller for the piston motor in the embodiment depicted in FIG. 3. Briefly, the code allows for programming of a volume and flow rate for each of the steps presented in FIGS. 4 through 8. The program includes first conversion of the volumes and flow rates to number of steps on the stepper motor and a given motor rate. The program then breaks the individual move steps of intake, filtration, backflush, and ejection into smaller moves. The smaller moves are generated to allow for compensation of the stepper rates based on the engagement of the piston inside the filter support grate. For example, at the initiation of the filtration step (e.g., FIG. 5(B)), the intake piston is outside of the filter support grate, while the dead-end piston is engaged in the filter support grate. As each individual step of the motor for each piston creates the same amount of vertical travel, but the effective cross-section of the fluid space is lower in the dead-end chamber, the dead-end piston must move at a faster rate to maintain the same change in volume, and subsequently, flow rate at the filter interface. The program also tracks where the system is in the process, and reports progress via a touchscreen, signal LEDs, or other mechanism (audible feedback, as an example). Lastly, the pseudocode includes safeguards to prevent the program from starting when there is no input syringe attached, or ejecting the volume when there is no output syringe attached to the device.

### Other Embodiments

While not depicted in the figures, other embodiments of the invention exist.

For example, the existing images depict a mechanically translated piston. However, a flexible membrane connecting the filter grate to a piston could be utilized to eliminate seal drag.

As discussed in the figures, a belted pulley system connected to motors is shown in the existing images. However, motors could be directly coupled to the threaded drive rod, could be coupled through gears or sprockets and chains, or other methods of coupling. Other devices such as hydraulic or pneumatic cylinders may be utilized.

The figures also depict a filter grate system supporting the filter. In an embodiment the filter media has a support structure integral to it. In such a case, there is no need for protrusions and recesses, so the piston can have a flat face.

In an embodiment the second filtration step requires forcing material across a semi-permeable/selectively permeable membrane. An embodiment uses two piston features on either side of the membrane in a sealed cylinder. The motion of the pistons draws the vacuum which pulls the solution into the filter chamber and across the filter, as well as controls the output of the cell concentrate into the output syringe. However, other methods for inducing this flow include elastic bellows type mechanisms in which a flexible outer surface is utilized instead of the rigid cylinder. Similar pistons are used, now with less drag, and create the same vacuum and pressure to move the fluid between the chambers.

An air bladder is used in some embodiments. It starts in an inflated position. As it is deflated via vacuum, it draws in the fluid. Likewise, the vacuum is reversed to fill the bladder again, forcing material out of the chamber. This would again be advantageous at eliminating drag of the piston on the cylinder wall.

Another embodiment includes a peristaltic pump which forces fluid through the filter and into a flexible chamber, presumably of a rubber type material. Upon complete filtration, a valve opens on the inlet side, and the pressurized chamber initiates the back flow to release the cellular content and flow material into an outlet chamber.

Other combinations of mechanically driven mechanisms to pressurize/induce vacuum to move fluid on either side of the permeable membrane could also be used.

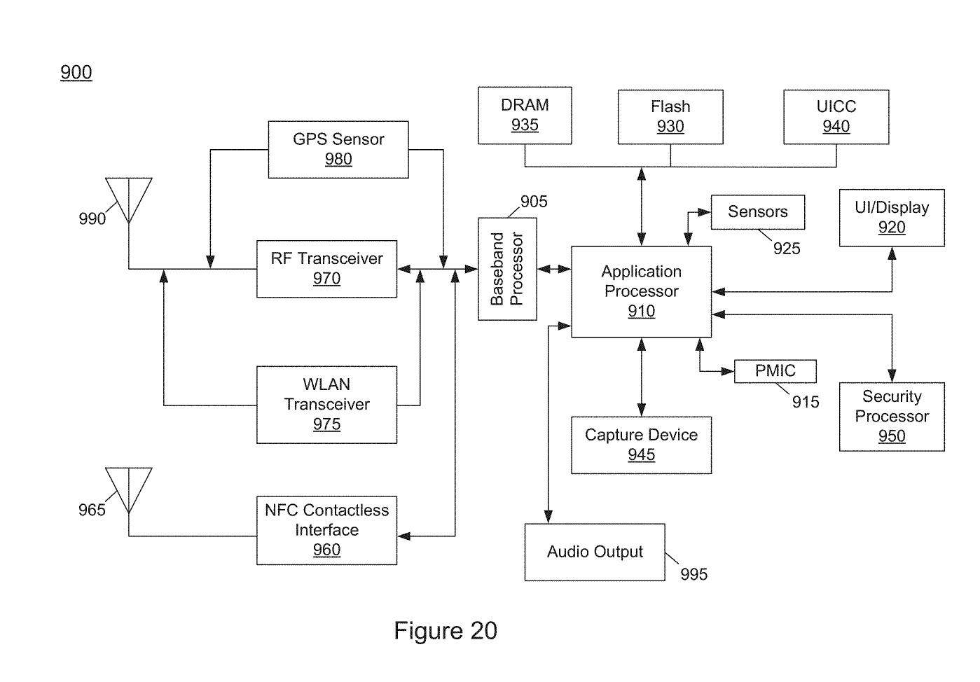

Referring now to FIG. 20, shown is a block diagram of an example system with which embodiments can be used. As seen, system 900 may be a smartphone or other wireless communicator or any other Internet of Things (IoT) device. A baseband processor 905 is configured to perform various signal processing with regard to communication signals to be transmitted from or received by the system. In turn, baseband processor 905 is coupled to an application processor 910 (e.g., a controller by Arduino), which may be a main CPU of the system to execute an OS and other system software, in addition to user applications such as an application to control a step motor for the filtration system described herein. Application processor 910 may further be configured to perform a variety of other computing operations for the device (e.g., pseudocode in FIGS. 19a-19g).

In turn, application processor 910 can couple to a user interface/display 920 (e.g., touch screen display). Such a display may provide prompts such as those describe in FIG. 18. In addition, application processor 910 may couple to a memory system including a non-volatile memory, namely a flash memory 930 and a system memory, namely a DRAM 935. As further seen, application processor 910 also couples to audio output 995 and a capture device 945 such as one or more image capture devices that can record video and/or still images.

A universal integrated circuit card (UICC) 940 comprises a subscriber identity module, which in some embodiments includes a secure storage to store secure user information. System 900 may further include a security processor 950 (e.g., Trusted Platform Module (TPM)) that may couple to application processor 910. A plurality of sensors 925, including one or more multi-axis accelerometers may couple to application processor 910 to enable input of a variety of sensed information such as motion and other environmental information (or other sensors such as phototransistors to monitor fluid flow or sensors to count a number of steps of a step motor). In addition, one or more authentication devices may be used to receive, for example, user biometric input for use in authentication operations.

As further illustrated, a near field communication (NFC) contactless interface 960 is provided that communicates in a NFC near field via an NFC antenna 965. While separate antennae are shown, understand that in some implementations one antenna or a different set of antennae may be provided to enable various wireless functionalities.

A power management integrated circuit (PMIC) 915 couples to application processor 910 to perform platform level power management. To this end, PMIC 915 may issue power management requests to application processor 910 to enter certain low power states as desired. Furthermore, based on platform constraints, PMIC 915 may also control the power level of other components of system 900.

To enable communications to be transmitted and received such as in one or more IoT networks, various circuitries may be coupled between baseband processor 905 and an antenna 990. Specifically, a radio frequency (RF) transceiver 970 and a wireless local area network (WLAN) transceiver 975 may be present. In general, RF transceiver 970 may be used to receive and transmit wireless data and calls according to a given wireless communication protocol such as 3G or 4G wireless communication protocol such as in accordance with a code division multiple access (CDMA), global system for mobile communication (GSM), long term evolution (LTE) or other protocol. In addition a GPS sensor 980 may be present, with location information being provided to security processor 950 for use as described herein when context information is to be used in a pairing process. Other wireless communications such as receipt or transmission of radio signals (e.g., AM/FM) and other signals may also be provided. In addition, via WLAN transceiver 975, local wireless communications, such as according to a Bluetooth™ or IEEE 802.11 standard can also be realized.

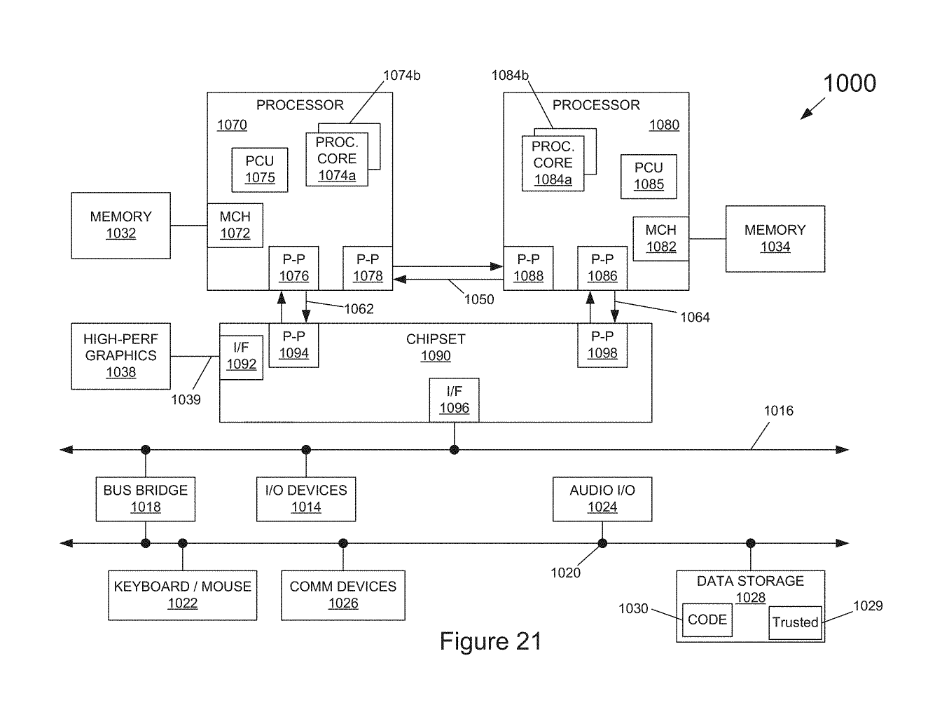

Referring now to FIG. 21, shown is a block diagram of a system in accordance with another embodiment of the present invention. Multiprocessor system 1000 is a point-to-point interconnect system such as a server system, and includes a first processor 1070 (e.g., a controller by Arduino) and a second processor 1080 coupled via a point-to-point interconnect 1050. Each of processors 1070 and 1080 may be multicore processors such as SoCs, including first and second processor cores (i.e., processor cores 1074a and 1074b and processor cores 1084a and 1088b), although potentially many more cores may be present in the processors. In addition, processors 1070 and 1080 each may include a secure engine 1075 and 1085 to perform security operations such as attestations, IoT network onboarding or so forth.

First processor 1070 further includes a memory controller hub (MCH) 1072 and point-to-point (P-P) interfaces 1076 and 1078. Similarly, second processor 1080 includes a MCH 1082 and P-P interfaces 1086 and 1088. MCH's 1072 and 1082 couple the processors to respective memories, namely a memory 1032 and a memory 1034, which may be portions of main memory (e.g., a DRAM) locally attached to the respective processors. First processor 1070 and second processor 1080 may be coupled to a chipset 1090 via P-P interconnects 1062 and 1064, respectively. Chipset 1090 includes P-P interfaces 1094 and 1098.

Furthermore, chipset 1090 includes an interface 1092 to couple chipset 1090 with a high performance graphics engine 1038, by a P-P interconnect 1039. In turn, chipset 1090 may be coupled to a first bus 1016 via an interface 1096. Various input/output (I/O) devices 1014 may be coupled to first bus 1016, along with a bus bridge 1018 which couples first bus 1016 to a second bus 1020. Various devices may be coupled to second bus 1020 including, for example, a keyboard/mouse 1022, communication devices 1026 and a data storage unit 1028 such as a non-volatile storage or other mass storage device. As seen, data storage unit 1028 may include code 1030, in one embodiment. As further seen, data storage unit 1028 also includes a trusted storage 1029 to store sensitive information to be protected. Further, an audio I/O 1024 may be coupled to second bus 1020.

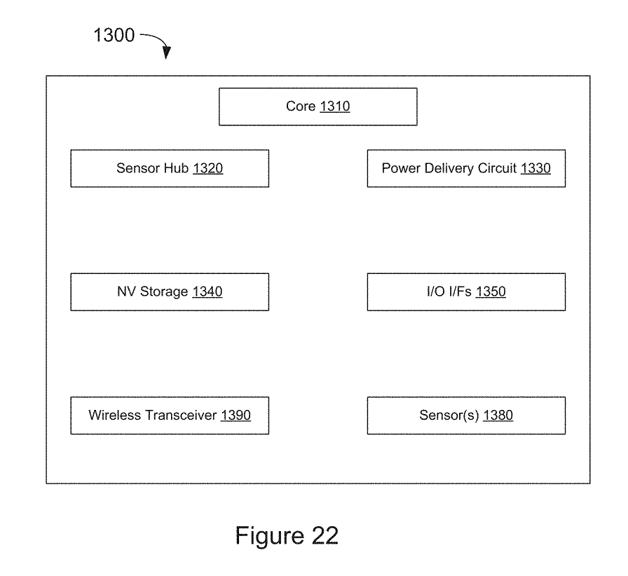

Referring now to FIG. 22, embodiments may be used in environments where IoT devices may include wearable devices or other small form factor IoT devices. Shown is a block diagram of a wearable module 1300 in accordance with another embodiment. In one particular implementation, module 1300 may be an Intel® Curie™ module that includes multiple components adapted within a single small module that can be implemented as all or part of a wearable device. As seen, module 1300 includes a core 1310 (of course in other embodiments more than one core may be present). Such core may be a relatively low complexity in-order core, such as based on an Intel Architecture® Quark™ design. In some embodiments, core 1310 may implement a Trusted Execution Environment (TEE). Core 1310 couples to various components including a sensor hub 1320, which may be configured to interact with a plurality of sensors 1380, such as one or more biometric, motion environmental or other sensors (e.g., sensors to measure fluid flow or step motor steps). A power delivery circuit 1330 is present, along with a non-volatile storage 1340. In an embodiment, this circuit may include a rechargeable battery and a recharging circuit, which may in one embodiment receive charging power wirelessly. One or more input/output (IO) interfaces 1350, such as one or more interfaces compatible with one or more of USB/SPI/12C/GPIO protocols, may be present. In addition, a wireless transceiver 1390, which may be a Bluetooth™ low energy or other short-range wireless transceiver is present to enable wireless communications as described herein. Understand that in different implementations a wearable module can take many other forms. Wearable and/or IoT devices have, in comparison with a typical general purpose CPU or a GPU, a small form factor, low power requirements, limited instruction sets, relatively slow computation throughput, or any of the above.

Embodiments may be used in many different types of systems. For example, in one embodiment a communication device can be arranged to perform the various methods and techniques described herein. Of course, the scope of the present invention is not limited to a communication device, and instead other embodiments can be directed to other types of apparatus for processing instructions, or one or more machine readable media including instructions that in response to being executed on a computing device, cause the device to carry out one or more of the methods and techniques described herein.

Program instructions may be used to cause a general-purpose or special purpose processing system that is programmed with the instructions to perform the operations described herein. Alternatively, the operations may be performed by specific hardware components that contain hardwired logic for performing the operations, or by any combination of programmed computer components and custom hardware components. The methods described herein may be provided as (a) a computer program product that may include one or more machine readable media having stored thereon instructions that may be used to program a processing system or other electronic device to perform the methods or (b) at least one storage medium having instructions stored thereon for causing a system to perform the methods. The term “machine readable medium” or “storage medium” used herein shall include any medium that is capable of storing or encoding a sequence of instructions (transitory media, including signals, or non-transitory media) for execution by the machine and that cause the machine to perform any one of the methods described herein. The term “machine readable medium” or “storage medium” shall accordingly include, but not be limited to, memories such as solid-state memories, optical and magnetic disks, read-only memory (ROM), programmable ROM (PROM), erasable PROM (EPROM), electrically EPROM (EEPROM), a disk drive, a floppy disk, a compact disk ROM (CD-ROM), a digital versatile disk (DVD), flash memory, a magneto-optical disk, as well as more exotic mediums such as machine-accessible biological state preserving or signal preserving storage. A medium may include any mechanism for storing, transmitting, or receiving information in a form readable by a machine, and the medium may include a medium through which the program code may pass, such as antennas, optical fibers, communications interfaces, and the like. Program code may be transmitted in the form of packets, serial data, parallel data, and the like, and may be used in a compressed or encrypted format. Furthermore, it is common in the art to speak of software, in one form or another (e.g., program, procedure, process, application, module, logic, and so on) as taking an action or causing a result. Such expressions are merely a shorthand way of stating that the execution of the software by a processing system causes the processor to perform an action or produce a result.

A module as used herein refers to any hardware, software, firmware, or a combination thereof. Often module boundaries that are illustrated as separate commonly vary and potentially overlap. For example, a first and a second module may share hardware, software, firmware, or a combination thereof, while potentially retaining some independent hardware, software, or firmware. In one embodiment, use of the term logic includes hardware, such as transistors, registers, or other hardware, such as programmable logic devices. However, in another embodiment, logic also includes software or code integrated with hardware, such as firmware or micro-code.

An embodiment utilizes two pistons to generate flow across a static filter, trapping the cellular content on the intake side of the device. There is no mixing of reagents and no moving pieces between the piston faces.

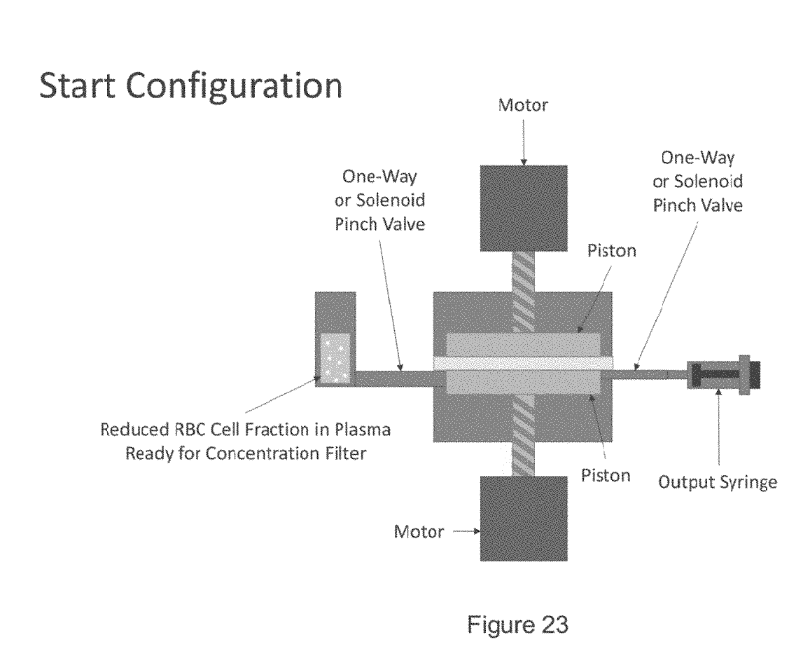

FIG. 23 depicts the initial configuration of the device. The pistons are fully seated against the filter material. There is no space for fluid in the chambers. The input cell suspension, aspirate with a reduced RBC fraction, is loaded into a holding vessel, connected by a hollow member to the intake chamber. A one way or solenoid valve is incorporated to prevent the solution from flowing back into the holding vessel. No fluid has entered the device at this time.

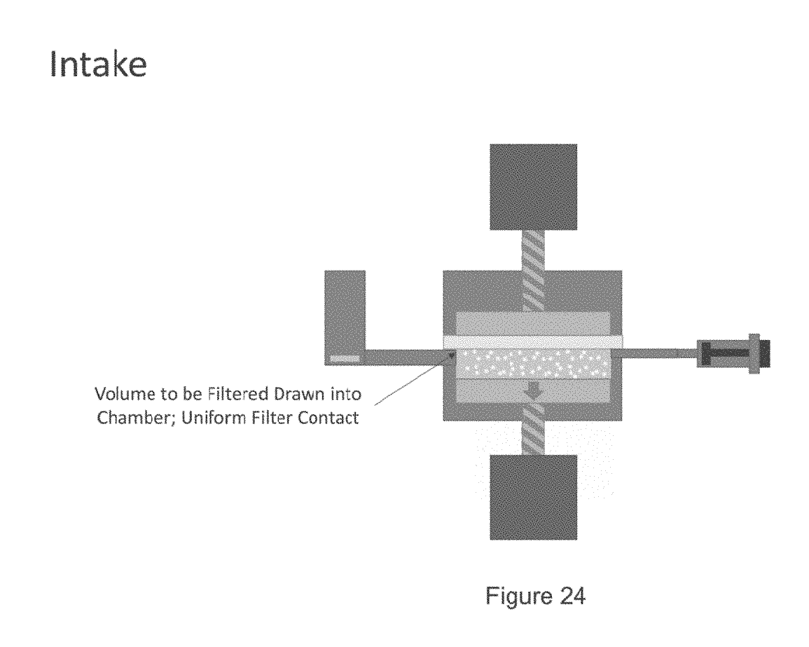

FIG. 24 depicts the intake step of the device. The intake piston has withdrawn to a position distal from the filter, increasing the volume, and creating a vacuum to draw the unfiltered fluid with cells into the intake reservoir. The dead-end piston remains fully seated against the filter material, and engagement of the piston face protrusions into the void spaces of the filter support grates.

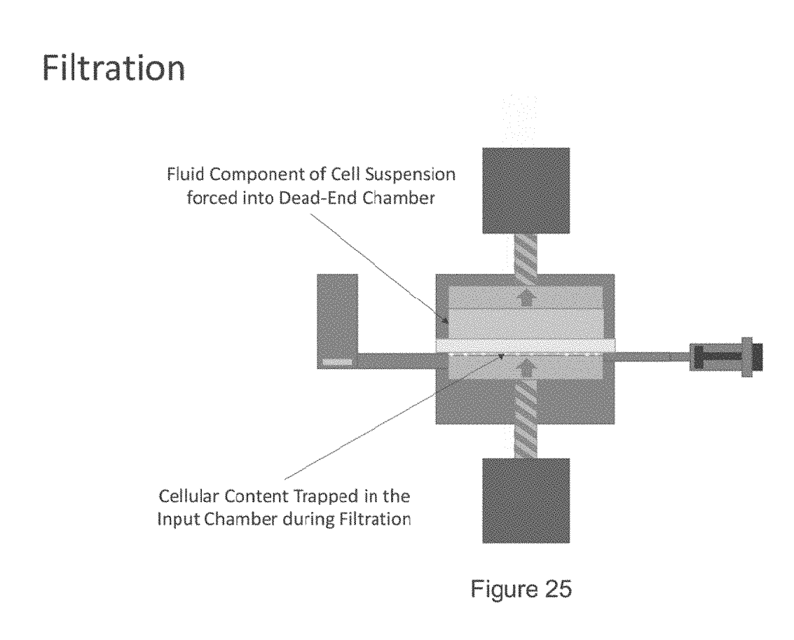

FIG. 25 depicts the filtration step of the device. The intake piston is moved to fully seat against the filter material, while the dead end piston has been simultaneously moved to a position distal to the filter. The overall volume between the two chambers is kept constant as the pistons move to prevent forcing fluid out of the chambers. The fluid is instead drawn through the filter and into the fluid trap. The cellular content is unable to move across the filter and is thus trapped on the intake side of the filter.

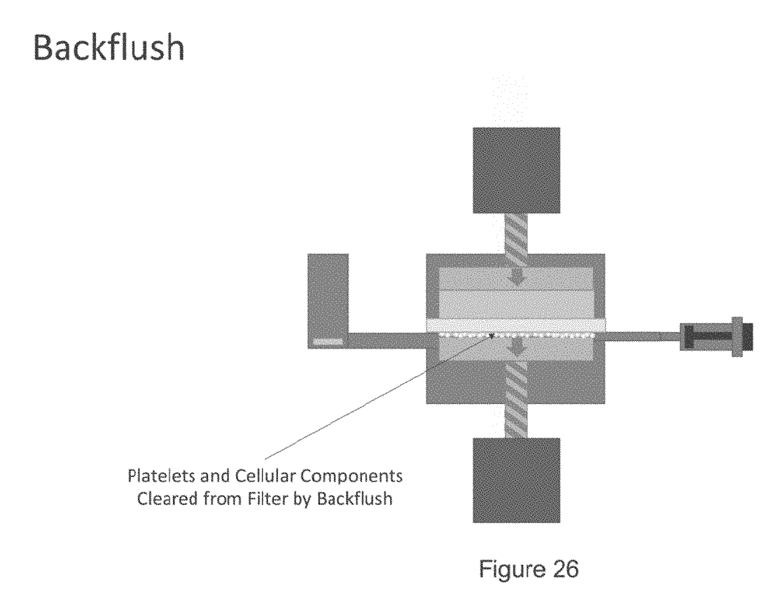

FIG. 26 depicts the backflush state of the device. The intake piston has been moved in a direction distal to the filter while the dead end piston is moved in a direction toward the filter medium. The magnitudes of the moves are a fraction of the movement of the initial intake volume. The cellular material is forced off of the filter and into suspension, but as the same cellular content is now in a smaller volume of fluid, the concentration of cells in the suspension has been increased. The combined volume of the two chambers has again been kept constant so that no fluid flows from the system.

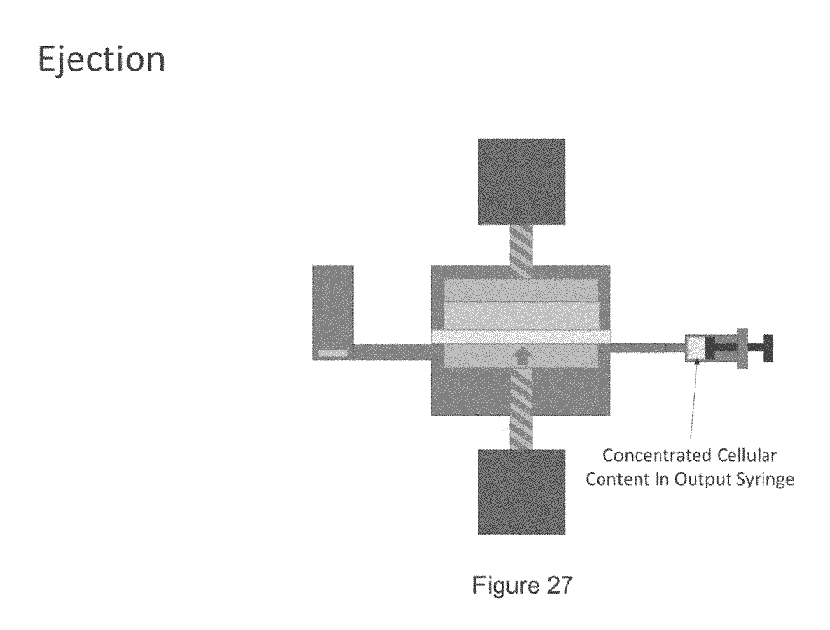

FIG. 27 depicts the ejection state of the device. The intake piston has moved toward the filter and is now fully seated against the filter material. The dead end piston retains the position it held at the backflush stage. The result is a reduction in volume of the system, which causes the suspension to de forced out of the system and into the awaiting output vessel.

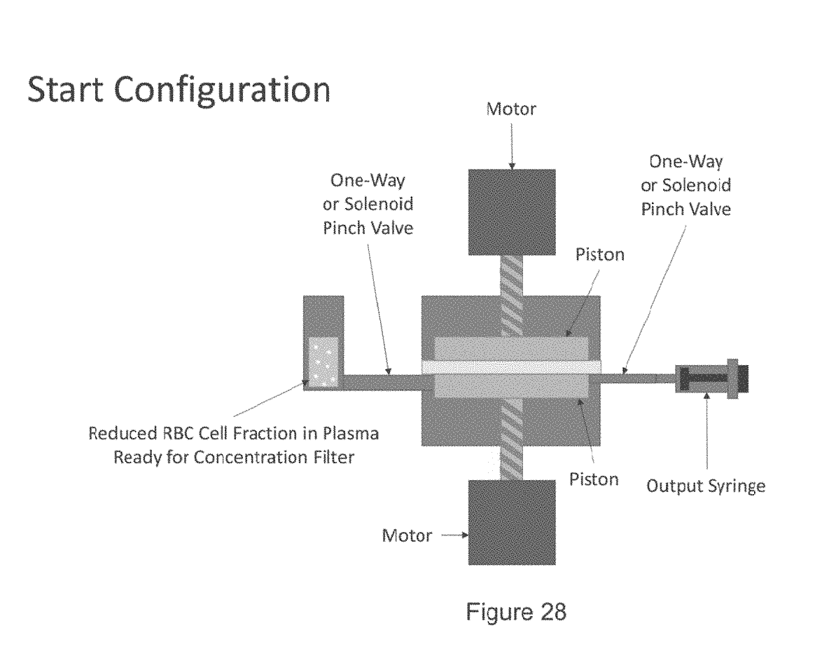

FIG. 28 depicts the initial configuration of the device in another embodiment. The pistons are fully seated against the filter material. There is no space for fluid in the chambers. The input cell suspension, aspirate with a reduced RBC fraction, is loaded into a holding vessel, connected by a hollow member to the intake chamber. A one way or solenoid valve is incorporated to prevent the solution from flowing back into the holding vessel. No fluid has entered the device at this time.

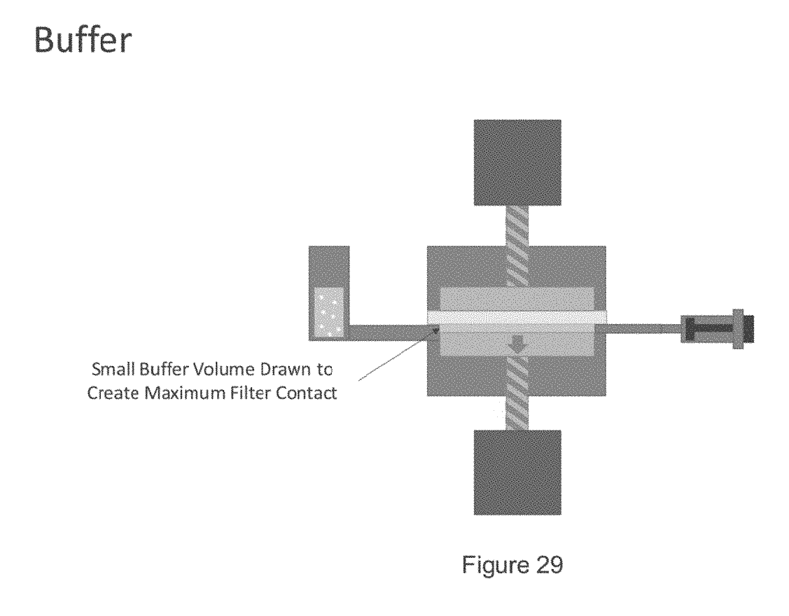

FIG. 29 depicts the device in the buffer. This is an alternative method for the filtration process. In this case, the intake side piston is partially withdrawn to draw a fraction of the unfiltered fluid with cells into the reservoir. This creates an even layer of fluid, distributed over the surface of the piston and allows full wetting of filter to occur without trapping air. Additionally, it allows filtration to occur over the entirety of the filter surface. The dead end piston remains fully seated against the filter material.

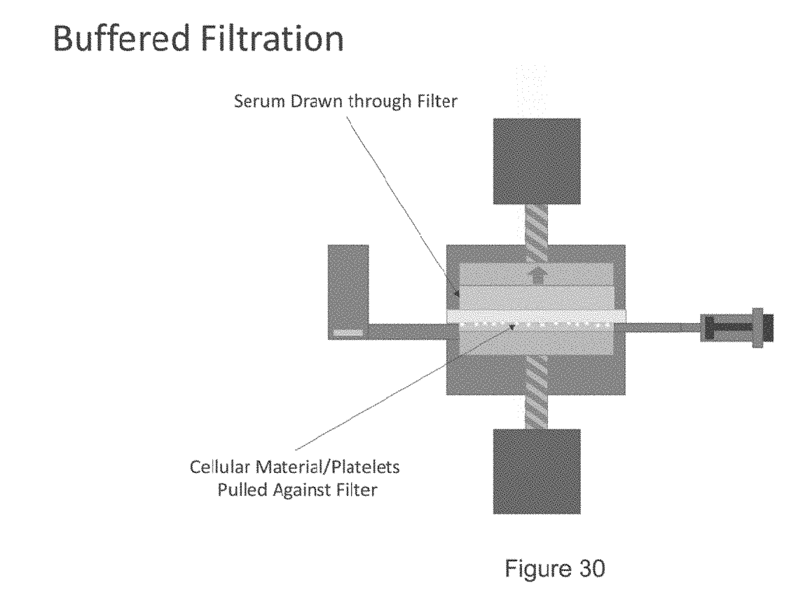

FIG. 30 depicts the device in the buffered filtration position. In this case, the intake side piston remains partially withdrawn to maintain a buffer space to allow for fluid flow that is not restricted as it would be when the intake piston is fully seated against the filter. The dead end piston has been withdrawn to pull the remainder of the fluid into the intake chamber and through the filter and into the fluid trap.

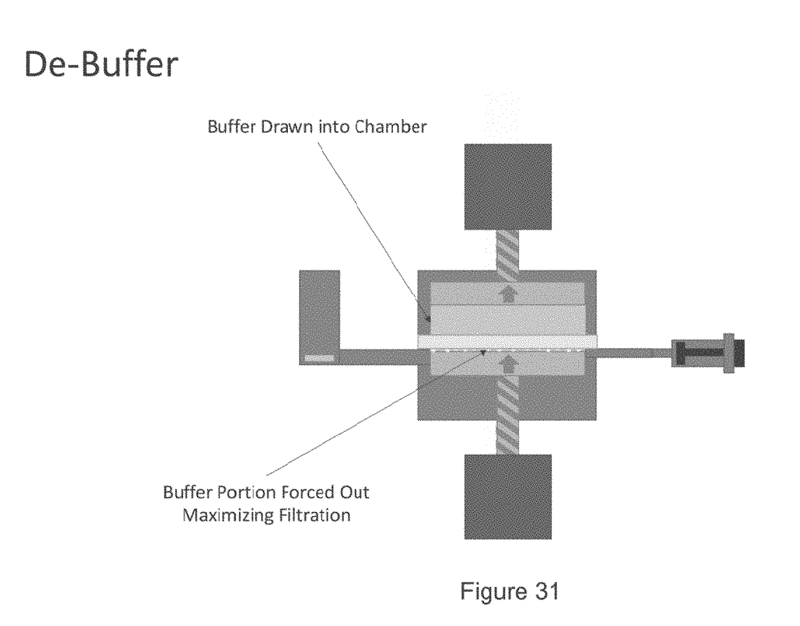

FIG. 31 depicts the device in the de-buffer position. The intake side piston has been fully seated against the filter material, while the dead end piston has been withdrawn to maintain the overall combined chamber volume. This causes the buffer portion of the fluid through the filter and into the dead end chamber, or fluid trap. In this state, all or most cellular material is trapped on the input side of the filter, while the mostly cell-free fluid portion is in the fluid trap in the dead end chamber.

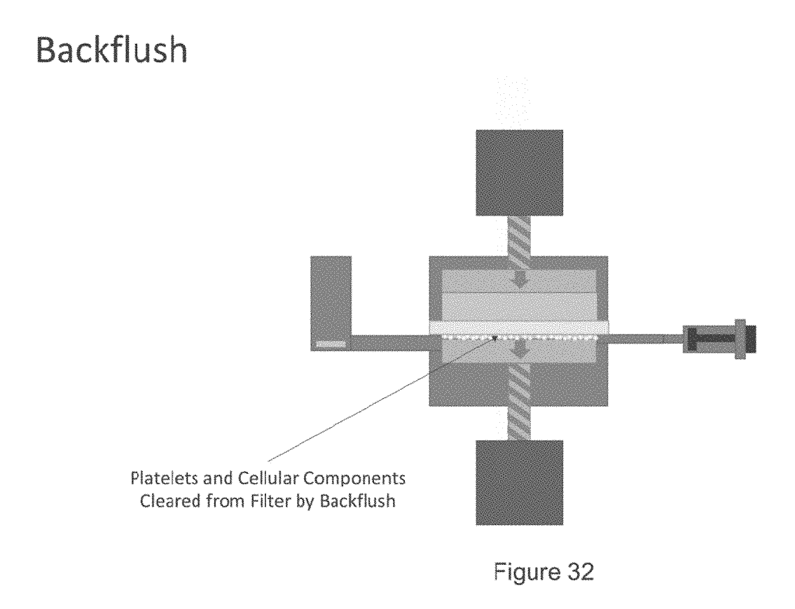

FIG. 32 depicts the backflush state of the device. The intake piston has been moved in a direction distal to the filter while the dead end piston is moved in a direction toward the filter medium. The magnitudes of the moves are a fraction of the movement of the initial intake volume. The cellular material is forced off of the filter and into suspension, but as the same cellular content is now in a smaller volume of fluid, the concentration of cells in the suspension has been increased. The combined volume of the two chambers has again been kept constant so that no fluid flows from the system.

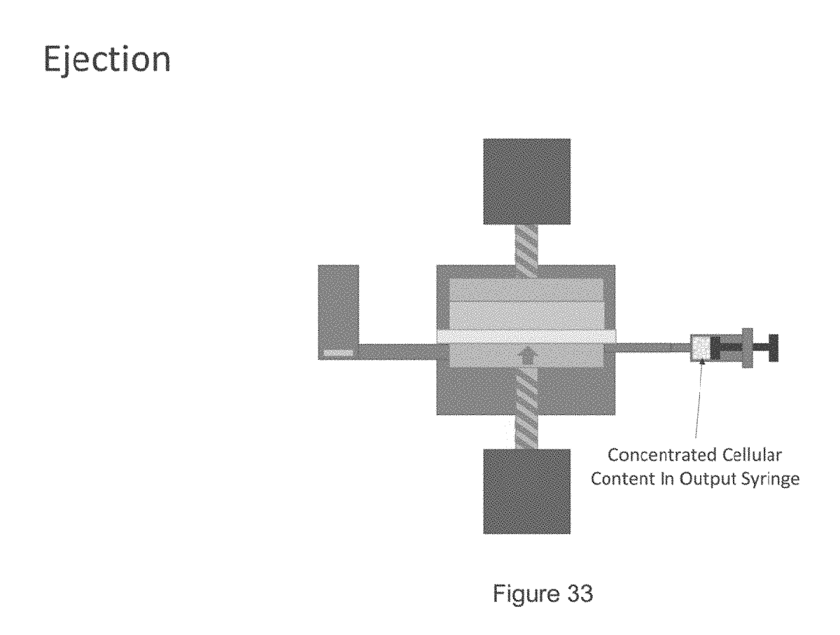

FIG. 33 depicts the ejection state of the device. The intake piston has moved toward the filter and is now fully seated against the filter material. The dead end piston retains the position it held at the backflush stage. The result is a reduction in volume of the system, which causes the suspension to be forced out of the system and into the awaiting output vessel.

The following examples pertain to further embodiments.

### EXAMPLE 1

A filtration system, composed of a filter separating two adjacent rigid chambers, each containing a mechanism to induce flow across the filter medium, the first rigid chamber including an opening (port) connected to a hollow member to allow fluid flow into and out of the first chamber.

### EXAMPLE 2

The system of Example 1 in which the first rigid chamber contains two openings or ports, both configured to allow fluid flow into and out of the first rigid chamber and whose connected hollow members are coupled to combine the fluid flow outside of the chamber.

### EXAMPLE 3

The system of Example 2 in which the first opening or port is configured for flow into the first rigid chamber and the second port is configured for flow out of the rigid chamber.

### EXAMPLE 4

The system of Example 1 in which the first rigid chamber contains multiple (greater than two) openings or ports, all configured to allow fluid flow into and out of the first rigid chamber and whose connected hollow members are coupled to combine the fluid flow outside of the chamber.

### EXAMPLE 5

The system of Example 4 in which a group one or more ports are configured only for flow into the first rigid chamber and a distinct separate group of one or more ports are configured for flow out of the first rigid chamber; each group having connected hollow members which are coupled to combine the fluid flow outside of the chamber.

### EXAMPLE 6

The system of Example 1 in which the ports are located on the rigid chamber.

### EXAMPLE 7

The system of Example 1 in which the ports are located on the flow inducing mechanism.

### EXAMPLE 8

The system of Example 1 in which the chamber containing ports or openings is located below the chamber that does not contain ports.

EXAMPLE 9

The system of example 1 in which the second chamber also contains one or more openings (ports) to allow fluid flow into and out of said second chamber.

### EXAMPLE 10

The system of Example 1 in which a mechanism is utilized to control fluid flow into and out of the chambers by opening or closing the ports and/or hollow members.

### EXAMPLE 11

The system of Example 10 in which the ports for the first chamber are regulated utilizing a passive one-way valve.

### EXAMPLE 12

The system of Example 10 in which the ports for the first chamber are regulated using a solenoid coupled to a pinch valve.

### EXAMPLE 13

The system of Example 10 in which the ports for the first chamber are regulated using a ball valve coupled to a rotary motor.

### EXAMPLE 14

The system of Example 1 in which the filter medium is supported by a rigid member containing a plurality of openings to allow for fluid flow across the filter while preventing the filter from bowing and/or tearing.

### EXAMPLE 15

The system of Example 14 in which a rigid member is used on each side if the filter media.

### EXAMPLE 16

The system of Example 14 in which the rigid member is incorporated into the filter media.

### EXAMPLE 17

The system of Example 1 in which the mechanisms to induce flow across the filter are pistons.

### EXAMPLE 18

The system of Example 17 in which the face of the piston(s) adjacent to the filter has a plurality of recesses to allow mating with the rigid member of Example 14.

### EXAMPLE 19

The system of Example 17 in which the piston contains features to maintain contact with the chamber wall to maintain parallelism between the piston face and the filter medium.

### EXAMPLE 20

The system of Example 17 in which the pistons have a threaded feature coupled to a threaded member driven by a hand crank.

### EXAMPLE 21

The system of Example 17 in which the pistons have a threaded feature coupled to a threaded member driven by a motor.

### EXAMPLE 22

The system of Example 21 in which a microcontroller is used to control the drive motors and subsequently, fluid flow into, out of, and between the chambers.

### EXAMPLE 23

The system of Example 17 in which the pistons are coupled to a hydraulic cylinder to drive the translational motion of the piston.

### EXAMPLE 24

The system of Example 23 in which a microcontroller is used to control the hydraulic cylinder and subsequently, fluid flow into, out of, and between the chambers.

### EXAMPLE 25

The system of Example 17 in which the pistons are coupled to a pneumatic cylinder to drive the translational motion of the piston.

### EXAMPLE 26

The system of Example 25 in which a microcontroller is used to control the pneumatic cylinder and subsequently, fluid flow into, out of, and between the chambers.

### EXAMPLE 27

The system of Example 1 in which the flow inducing member is a pre-inflated air bladder which can be deflated to create a vacuum and induce flow into a chamber, or inflated to create pressure, inducing flow out of a chamber.

### EXAMPLE 28

A method for utilizing the system of Example 1 in which: a first solution containing a first concentration of cells is flowed into a first chamber, separated from a second chamber by a filter medium; the fluid component of the first solution is then forced through the filter, resulting in a second solution in the second chamber, opposite of the filter, which has little to no cellular concentration; a smaller volume of the second solution is forced across the filter in the opposite direction, to dislodge cellular content trapped on the filter media into the first solution, resulting in a third solution, which has a higher cellular concentration than the first; the third, concentrated solution is forced out of the system into an output reservoir for end use.

### EXAMPLE 29

The method of Example 28 in which: a fraction of the first solution containing a first concentration of cells is flowed into a first chamber, separated from a second chamber by a filter medium; the fluid component of the remaining volume of the first solution is then forced through the filter, resulting in a second solution in the second chamber, opposite of the filter, which has little to no cellular concentration; the initial buffer volume of the first solution is forced into the second chamber; a smaller volume of the second solution is forced across the filter in the opposite direction, to dislodge cellular content trapped on the filter media into the first solution, resulting in a third solution, which has a higher cellular concentration than the first; the third, concentrated solution is force out of the system into an output reservoir for end use.

For example, the steps of example 29 may occur before the steps of example 28. However, in other embodiments the steps of example 28 may occur in lieu of the steps of example 29. In other words, in an embodiment the buffer related actions (e.g., FIGS. 9-11) may occur in lieu of the first method (e.g., FIGS. 4-9). Essentially, the buffer method allows for the filter process to run faster as the whole volume to be filtered is not drawn into the intake chamber first. However, in situations with highly viscous fluids, or extremely high cell concentrations, the first method may be implemented.

### EXAMPLE 30

The method of Example 28 in which a microcontroller is used to regulate the flow of each portion of the method by controlling a motor, hydraulic cylinder, pneumatic cylinder, or other drive method.

### EXAMPLE 31

The method of Example 30 in which the microcontroller compensates for differences in volumes inside and outside of a filter supporting member to maintain a constant flow rate.

### EXAMPLE 32

The system of Example 1 in which the chambers are formed from an elastic material which deforms to create the chamber without introducing drag on the mechanisms configured to draw fluid into the chamber.

### EXAMPLE 33

The system of Example 32, in which the filter is supported by a rigid member on either side of the filter medium, each rigid member containing a plurality of openings to allow fluid flow through the filter medium.

### EXAMPLE 34

The system of example 32 in which the flexible chamber is expanded and contracted utilizing a mechanical attachment to the flexible member.

### EXAMPLE 35

The system of example 32 in which the flexible chamber is expanded and contracted utilizing a pump to force material into and out of each chamber.

### EXAMPLE 36

The system of Example 32 in which the flexible chamber is connected to a piston, resulting in chamber with a rigid surface on the chamber opposite the filter, and an elastic surface on the peripheral walls of the chamber.

### EXAMPLE 37

The system of Example 36 in which the face of the piston(s) adjacent to the filter has a plurality of recesses to allow mating with the rigid member of Example 33.

### EXAMPLE 38

The system of Example 36 in which the pistons have a threaded feature coupled to a threaded member driven by a hand crank.

### EXAMPLE 39

The system of Example 36 in which the pistons have a threaded feature coupled to a threaded member driven by a motor.

### EXAMPLE 40

The system of Example 39 in which a microcontroller is used to control the drive motors and subsequently, fluid flow into, out of, and between the chambers.

### EXAMPLE 41

The system of Example 36 in which the pistons are coupled to a hydraulic cylinder to drive the translational motion of the piston.

### EXAMPLE 42

The system of Example 41 in which a microcontroller is used to control the hydraulic cylinder and subsequently, fluid flow into, out of, and between the chambers.

### EXAMPLE 43

The system of Example 36 in which the pistons are coupled to a pneumatic cylinder to drive the translational motion of the piston.

### EXAMPLE 44

The system of Example 43 in which a microcontroller is used to control the pneumatic cylinder and subsequently, fluid flow into, out of, and between the chambers.

### EXAMPLE 45

The system of Example 32 in which a pump drives fluid into an area outside of the first and second chambers to generate motion of the piston without mechanical contact.

### EXAMPLE 46

The system of Example 1 in which fluid is driven by a pump.

### EXAMPLE 47

A method executed by at least one processor comprising: a first solution containing a first concentration of cells is flowed into a first chamber, separated from a second chamber by a filter medium; the fluid component of the first solution is then forced through the filter, resulting in a second solution in the second chamber, opposite of the filter, which has little to no cellular concentration; a smaller volume of the second solution is forced across the filter in the opposite direction, to dislodge cellular content trapped on the filter media into the first solution, resulting in a third solution, which has a higher cellular concentration than the first; the third, concentrated solution is forced out of the system into an output reservoir for end use.

### EXAMPLE 48

At least one machine readable medium comprising a plurality of instructions that in response to being executed on a computing device, cause the computing device to carry out a method according to examples 28.

### EXAMPLE 49

An apparatus comprising means for performing example 28.

### EXAMPLE 1a

A blood filtering system comprising: a first piston included in a first chamber; a second piston included in a second chamber; a filter between the first and second pistons; at least one memory and at least one processor, coupled to the at least one memory, to perform operations comprising: move the first piston away from the filter, while the second piston is still, to advance blood into the first chamber from a first container; move the first piston towards the filter while simultaneously moving the second piston away from the filter to advance blood from the first chamber across the filter and into the second chamber and to locate cells of the blood against the filter and within the first chamber; move the first piston away from the filter while simultaneously moving the second piston towards the filter to advance blood from the second chamber across the filter and into the first chamber and to flush the cells of the blood off of the filter; move the first piston toward the filter, while the second piston is still, to advance the cells of the blood out of the first chamber and into a second container.

### EXAMPLE 2a

The system of example 1a comprising: a first grate, included in the first chamber between the filter and the first piston, configured to support the filter; a second grate, included in the second chamber between the filter and the second piston, configured to support the filter; wherein the first grate has first grate protrusions arranged in a first grate pattern; and wherein the second grate has second grate protrusions arranged in a second grate pattern.

### EXAMPLE 3a

The system of example 2a wherein: the first piston has a first piston face having first piston protrusions arranged in a first piston pattern so the first piston protrusions fit between the first grate protrusions when the first piston is immediately adjacent the filter; the second piston has a second piston face having second piston protrusions arranged in a second piston pattern so the second piston protrusions fit between the second grate protrusions when the second piston is immediately adjacent the filter.

### EXAMPLE 4a

The system of example 3a wherein: the first piston face has first voids between the first piston protrusions; the second piston face has second voids between the second piston protrusions; a first void of the first voids includes a first aperture; the first aperture is coupled to a first channel that is to fluidly couple to the second container.

### EXAMPLE 5a

The system of example 4a comprising a second aperture, wherein: the second aperture is coupled to a second channel that is to fluidly couple to the second container; a second void of the first voids includes a second aperture.

### EXAMPLE 6a

The system of example 3a wherein: the filter is substantially located in a plane; one of the first grate protrusions extends away from the filter, in a direction orthogonal to the plane, a first distance; the one of the first grate protrusions and another of the first grate protrusions define a void that extends away from the filter the first distance; the at least on processor is to perform operations comprising: move, at a first rate of speed, the first piston towards the filter but before the first piston is included in the void; move, at a second rate of speed, the first piston towards the filter while the first piston is included in the void; wherein the second rate of speed is faster than the first rate of speed.

### EXAMPLE 7a

The system of example 3a comprising a first conduit, wherein: the first conduit is to couple the first container to the first chamber; the first conduit includes a first output located on the first piston face.

### EXAMPLE 8a

The system of example 7a comprising a second conduit, wherein: the second conduit is to couple the first container to the first chamber; the second conduit includes a second output located on the first piston face.

### EXAMPLE 9a

The system of example 2a comprising: at least one motor coupled to at least one processor; wherein the at least one motor is to move the first piston within the first chamber in response to first commands from the at least one processor; wherein the at least one motor is to move the second piston within the second chamber in response to second commands from the at least one processor.

### EXAMPLE 10a

The system of example 9a wherein the at least one motor is move the first piston independently of moving the second piston.

### EXAMPLE 11a

The system of example 9a wherein: the at least one motor includes a step motor; the at least one processor is to perform operations comprising: convert a number of steps to be taken by the step motor to at least one of a volume or a flow rate; track a number of steps taken by the step motor to determine a location of the first piston; rotate a portion of the step motor clockwise to move the first piston in a first direction; rotate the portion of the step motor counterclockwise to move the first piston in a second direction which is opposite the first direction.

### EXAMPLE 12a

The system of example 11a wherein: the first piston has a maximum range of motion within the first chamber; a plurality of steps by the step motor is needed to move the first piston across the maximum range of motion.

### EXAMPLE 13a

The system of example 2a wherein: the first piston has a first maximum range of motion within the first chamber; moving the first piston away from the filter, while the second piston is still, to advance blood into the first chamber from the first container includes moving the first piston away from the filter but not across the first maximum range of motion; the at least one processor is to perform operations comprising, after moving the first piston away from the filter, moving the second piston away from the filter, while the first piston is still, to advance blood into the second chamber from the first.

### EXAMPLE 14a

The system of example 13a wherein: the second piston has a second maximum range of motion within the second chamber; the first grate is to simultaneously directly contact both the filter and first piston when the first piston is located at one end of the first maximum range of motion; and the second grate is to simultaneously directly contact both the filter and second piston when the second piston is located at one end of the second maximum range of motion.

### EXAMPLE 15a

The system of example 2a wherein the at least on processor is to perform operations comprising: move, at a first rate of speed, the first piston towards the filter while simultaneously moving the second piston away from the filter to advance blood from the first chamber across the filter and into the second chamber and to locate the cells of the blood against the filter and within the first chamber; move, at a second rate of speed, the first piston away from the filter while simultaneously moving the second piston towards the filter to advance the blood from the second chamber across the filter and into the first chamber and to flush the cells of the blood off of the filter; wherein the second rate of speed is faster than the first rate of speed.

### EXAMPLE 16a

The system of example 1a wherein: the first piston has a maximum range of motion within the first chamber, the maximum range extending from a first portion directly adjacent the filter to a second portion at an opposite end of the maximum range of motion from the first portion; the at least one processor is to perform operations comprising moving the first piston toward the filter starting from a location not included in the second portion and, as a result, moving the first piston toward the filter across less than the maximum range of motion to advance the cells of the blood out of the first chamber and into the second container.

### EXAMPLE 17a

The system of example 1a, wherein the system does not include a centrifuge.

### EXAMPLE 1b

A blood filtering system comprising: a first piston included in a first chamber; a second piston included in a second chamber; a filter between the first and second pistons; at least one memory and at least one processor, coupled to the at least one memory, to perform operations comprising: move the first piston away from the filter, while the second piston is still, to advance blood into the first chamber from a first container; move the first piston towards the filter while simultaneously moving the second piston away from the filter to advance blood from the first chamber across the filter and into the second chamber and to locate cells of the blood against the filter and within the first chamber; move the first piston away from the filter while simultaneously moving the second piston towards the filter to advance blood from the second chamber across the filter and into the first chamber and to flush the cells of the blood off of the filter; move the first piston toward the filter, while the second piston is still, to advance the cells of the blood out of the first chamber and into a second container.

While the above example provides a system with a hardware filtering system (including pistons and a filter) and a processor and memory, other embodiments may include subsets of the system. For example, an embodiment may include the hardware filtering system without the processor and memory. An embodiment may just include the memory and code with which to drive the filter hardware. The memory may be cloud-based memory.

Another version of Example 1b. A blood filtering system comprising: a first piston included in a first chamber; a second piston included in a second chamber; a filter between the first and second pistons; a machine-readable medium having stored thereon data, which if used by at least one machine, causes the at least one machine to perform a method comprising: move the first piston away from the filter, while the second piston is still, to advance blood into the first chamber from a first container; move the first piston towards the filter while simultaneously moving the second piston away from the filter to advance blood from the first chamber across the filter and into the second chamber and to locate cells of the blood against the filter and within the first chamber; move the first piston away from the filter while simultaneously moving the second piston towards the filter to advance blood from the second chamber across the filter and into the first chamber and to flush the cells of the blood off of the filter; move the first piston toward the filter, while the second piston is still, to advance the cells of the blood out of the first chamber and into a second container.

### EXAMPLE 2b

The system of example 1b comprising: a first grate, between the filter and the first piston, to support the filter; and a second grate, between the filter and the second piston, to support the filter; wherein the first grate has first grate protrusions arranged in a first grate pattern; wherein the second grate has second grate protrusions arranged in a second grate pattern.

For instance, the first and second grates operate to keep the filter, which may be a thin membrane, from excessive warping or pending during fluid flow across the filter and/or during vacuum generation within one or both of the first and second chambers. This stability promotes better filtering of cells.

### EXAMPLE 3b

The system of example 2b wherein: the first piston has a first maximum range of motion within the first chamber; the second piston has a second maximum range of motion within the second chamber; the first piston has a first piston face having first piston protrusions arranged in a first piston pattern so the first piston protrusions fit between the first grate protrusions when the first piston is at one end of the first maximum range of motion; the second piston has a second piston face having second piston protrusions arranged in a second piston pattern so the second piston protrusions fit between the second grate protrusions when the second piston is at one end of the second maximum range of motion.

For instance, the piston protrusions may be keyed to the grate protrusions. An example of a piston protrusion is element 1301 of FIG. 13a. An example of a grate void (formed by grate protrusions) is void 1402 formed by grate protrusion 1401 of FIG. 14a.

### EXAMPLE 4b

The system of example 3b wherein: the first piston face has first voids between the first piston protrusions; a first void of the first voids includes a first aperture; the first aperture is coupled to a first channel that is to fluidly couple to the second container.

### EXAMPLE 5b

The system of example 4b comprising a second aperture, wherein: a second void of the first voids includes a second aperture; the second aperture is coupled to a second channel that is to fluidly couple to the second container.

For instance, see FIG. 17a and element 1702, 1703, 1704. Locating output ports in the recesses may be better collect cells distributed across the filter.

### EXAMPLE 6b

The system of example 3b wherein: the filter is substantially located in a plane; one of the first grate protrusions extends away from the filter, in a direction orthogonal to the plane, a first distance; the one of the first grate protrusions and another of the first grate protrusions define a void that extends away from the filter the first distance; the at least one processor is to perform operations comprising: move, at a first rate of speed, the first piston towards the filter but before the first piston is included in the void; move, at a second rate of speed, the first piston towards the filter while the first piston is included in the void; wherein the second rate of speed is faster than the first rate of speed.

For instance, and as mentioned above, at the initiation of the filtration step (e.g., FIG. 5(B)), the intake piston is outside of the filter support grate, while the dead-end piston is engaged in the filter support grate. As each individual step of the motor for each piston creates the same amount of vertical travel, but the effective cross-section of the fluid space is lower in the dead-end chamber, the dead-end piston must move at a faster rate to maintain the same change in volume, and subsequently, flow rate at the filter interface.

### EXAMPLE 7b

The system of example 3b comprising a first conduit, wherein: the first conduit is to couple the first container to the first chamber; the first conduit includes a first output located on the first piston face.

For instance, by drawing the solution in from a location distal (below) to the filter medium, the cell suspension may avoid contact with the filter medium until the chamber is filled (and therefore any trapped air or gas may move to the top of the dead-end chamber 908). A goal of the distal location is to avoid wetting (fluid contact) of the filter as it becomes impermeable to gas once wetted in some embodiments.

### EXAMPLE 8b

The system of example 7b comprising a second conduit, wherein: the second conduit is to couple the first container to the first chamber; the second conduit includes a second output located on the first piston face.

For instance, by increasing the number of inputs the cells may be better distributed across the piston and eventually the filter.

### EXAMPLE 9b

The system of example 2b comprising: at least one motor coupled to the at least one processor; wherein the at least one motor is to move the first piston within the first chamber in response to first commands from the at least one processor; wherein the at least one motor is to move the second piston within the second chamber in response to second commands from the at least one processor.

In some embodiments a single motor may move both the first and second pistons, while in other embodiments a motor may move the first piston and another motor may move the second piston. In other embodiments, multiple systems such as the system of FIG. 1 may be operated in parallel. For example, to speed the process a user may fill three syringes with BMA and couple the three syringes to three systems (such as the system of FIG. 1) in parallel to increase processing efficiency. A motor may drive each of the three systems. The motor may drive each lower piston towards the filter (to filter the BMA) simultaneously or in serial fashion.

### EXAMPLE 10b

The system of example 9b wherein the at least one motor is move the first piston independently of moving the second piston.

For instance, one piston may be moving while the other piston is still.

### EXAMPLE 11b

The system of example 9b wherein: the at least one motor includes a step motor; the at least one processor is to perform operations comprising: convert a number of steps to be taken by the step motor to at least one of a volume or a flow rate; track a number of steps taken by the step motor to determine a location of the first piston; rotate a portion of the step motor clockwise to move the first piston in a first direction; rotate the portion of the step motor counterclockwise to move the first piston in a second direction which is opposite the first direction.

### EXAMPLE 12b

The system of example 11b wherein: the first piston has a maximum range of motion within the first chamber; a plurality of steps by the step motor is needed to move the first piston across the maximum range of motion. For instance, a maximum range of motion for the input piston 501 may be from the filter 502 to the lowest point in the input chamber 503 (i.e., towards bottom of FIG. 5b) that the input piston face reaches during the entire filtering sequence addressed in FIGS. 5a to 8a.

### EXAMPLE 13b

The system of example 2b wherein: the first piston has a first maximum range of motion within the first chamber; moving the first piston away from the filter, while the second piston is still, to advance blood into the first chamber from the first container includes moving the first piston away from the filter but not across the first maximum range of motion; the at least one processor is to perform operations comprising, after moving the first piston away from the filter, moving the second piston away from the filter, while the first piston is still, to advance blood into the second chamber from the first container.

For instance, this may concern buffering as addressed with FIGS. 9b-10b.

### EXAMPLE 14b

The system of example 13b wherein: the second piston has a second maximum range of motion within the second chamber; the first grate is to simultaneously and directly contact both the filter and the first piston when the first piston is located at one end of the first maximum range of motion; and the second grate is to simultaneously and directly contact both the filter and second piston when the second piston is located at one end of the second maximum range of motion.

For instance, if a film is on the piston face or grate the film would not prevent the first grate from simultaneously and directly contacting both the filter and the first piston when the first piston is located at one end of the first maximum range of motion.

### EXAMPLE 15b

The system of example 2b wherein the at least one processor is to perform operations comprising: move, at a first rate of speed, the first piston towards the filter while simultaneously moving the second piston away from the filter to advance blood from the first chamber across the filter and into the second chamber and to locate the cells of the blood against the filter and within the first chamber; move, at a second rate of speed, the first piston away from the filter while simultaneously moving the second piston towards the filter to advance the blood from the second chamber across the filter and into the first chamber and to flush the cells of the blood off of the filter; wherein the second rate of speed is faster than the first rate of speed.

For instance, this allows a movement closer to a “pulse” to eject cells forcibly from the membrane without having to pass excess fluid from the upper chamber to the lower chamber. See above discussion regarding FIGS. 7a and 7b.

### EXAMPLE 16b

The system of example 1b wherein: the first piston has a maximum range of motion within the first chamber, the maximum range extending from a first portion of the maximum range of motion directly adjacent the filter to a second portion of the maximum range of motion at an opposite end of the maximum range of motion from the first portion of the maximum range of motion; the at least one processor is to perform operations comprising moving the first piston toward the filter starting from a location not included in the second portion of the maximum range of motion and, as a result, moving the first piston toward the filter across less than the maximum range of motion to advance the cells of the blood out of the first chamber and into the second container.

As mentioned above, FIGS. 7a and 7b depict the backflush state of the device, when the fluid component of the initial cell suspension is forced through the filter in a reverse direction, in detailed and simplified forms, respectively. In each case, intake piston 701 is displaced in a direction away from the filter medium 702, while the dead-end piston 703 is moved in a direction toward the filter medium. The magnitude of the movement in this step is a fraction (half or less) of the movement of the filtration step (FIGS. 6a and 6b). As the filter chambers are sealed, pressure is created in the dead-end chamber 704, while the volume of the intake chamber 705 is increased to create a vacuum and space for the fluid to flow. By maintaining the total overall volume in the system, no fluid enters or leaves the system. This motion induces flow of the fluid component 706 backward through the filter. The cellular content which was previously trapped and deposited on the surface of the filter 707 is forced back into the suspension 708 on the intake side. As a smaller volume of the fluid component is moved into the intake chamber, with the same cellular content (as the filter prevented the cells from flowing into the dead-end chamber, the resulting concentration of the cell suspension is greater than the original un-concentrated cell suspension).

Thus, the “smaller volume” leads to a highly concentrated cell suspension.

### EXAMPLE 17b

The system of example 1b, wherein the system does not include a centrifuge.

### EXAMPLE 1c

A blood filtering system comprising: a first piston included in a first chamber; a second piston included in a second chamber; a filter between the first and second pistons; at least one memory and at least one processor, coupled to the at least one memory, to perform operations comprising: move the first piston away from the filter, while the second piston is still, to advance bone marrow aspirate (BMA) into the first chamber from a first container; move the first piston towards the filter while simultaneously moving the second piston away from the filter to: (a)(i) advance a fluid fraction of the BMA from the first chamber across the filter and into the second chamber, and (a)(ii) locate a cellular fraction of the BMA adjacent the filter and within the first chamber; move the first piston away from the filter while simultaneously moving the second piston towards the filter to advance the fluid fraction from the second chamber across the filter and into the first chamber and to flush the cellular fraction away from the filter; move the first piston toward the filter, while the second piston is still, to advance the cellular fraction out of the first chamber and into a second container.

For instance, “locate a cellular fraction of the BMA adjacent the filter” may mean locating the cellular fraction within a void 1402 of a grate. In other embodiments adjacent may mean within 2mm of the filter. A goal of various embodiments is to deliver more cellular fraction and less fluid fraction to a medical care provider. Consequently, adjacent would mean the cellular fraction is very close to the filter to reduce the fluid fraction but does not necessarily have to contact the filter.

An example of a filter may include the Vivid Plasma Separation membrane (which includes Asymmetric polysulfone) available from Pall Corporation.

### EXAMPLE 2c

The system of example 1c comprising: a first grate, between the filter and the first piston, to support the filter; and a second grate, between the filter and the second piston, to support the filter; wherein the first grate has first grate protrusions arranged in a first grate pattern; wherein the second grate has second grate protrusions arranged in a second grate pattern.

### EXAMPLE 3c

The system of example 2c wherein: the first piston has a first maximum range of motion within the first chamber; the second piston has a second maximum range of motion within the second chamber; the first piston has a first piston face having first piston protrusions arranged in a first piston pattern so the first piston protrusions fit between the first grate protrusions when the first piston is at one end of the first maximum range of motion; the second piston has a second piston face having second piston protrusions arranged in a second piston pattern so the second piston protrusions fit between the second grate protrusions when the second piston is at one end of the second maximum range of motion.

### EXAMPLE 4c

The system of example 3c wherein: the first piston face has first voids between the first piston protrusions; a first void of the first voids includes a first aperture; the first aperture is coupled to a first channel that is to fluidly couple to the second container.

### EXAMPLE 5c

The system of example 4c comprising a second aperture, wherein: a second void of the first voids includes a second aperture; the second aperture is coupled to a second channel that is to fluidly couple to the second container.

### EXAMPLE 6c

The system of example 3c wherein: the filter is substantially located in a plane; one of the first grate protrusions extends away from the filter, in a direction orthogonal to the plane, a first distance; the one of the first grate protrusions and another of the first grate protrusions define a void that extends away from the filter the first distance; the at least one processor is to perform operations comprising: move, at a first rate of speed, the first piston towards the filter but before the first piston is included in the void; move, at a second rate of speed, the first piston towards the filter while the first piston is included in the void; wherein the second rate of speed is faster than the first rate of speed.

Another version of Example 6c. The system of example 3c wherein: the at least one processor is to perform operations comprising: move, at a first rate of speed, the first piston towards the filter but before the first piston is between two or more first gate protrusions; move, at a second rate of speed, the first piston towards the filter while the first piston is between two or more gate protrusion; wherein the second rate of speed is faster than the first rate of speed.

### EXAMPLE 7c

The system of example 3c comprising a first conduit, wherein: the first conduit is to couple the first container to the first chamber; the first conduit includes a first output located on the first piston face.

### EXAMPLE 8c

The system of example 7c comprising a second conduit, wherein: the second conduit is to couple the first container to the first chamber; the second conduit includes a second output located on the first piston face.

### EXAMPLE 9c

The system of example 2c comprising: at least one motor coupled to the at least one processor; wherein the at least one motor is to move the first piston within the first chamber in response to first commands from the at least one processor; wherein the at least one motor is to move the second piston within the second chamber in response to second commands from the at least one processor.

### EXAMPLE 10c

The system of example 9c wherein the at least one motor is move the first piston independently of moving the second piston.

### EXAMPLE 11c

The system of example 9c wherein: the at least one motor includes a step motor; the at least one processor is to perform operations comprising: convert a number of steps to be taken by the step motor to at least one of a volume or a flow rate; and track a number of steps taken by the step motor to determine a location of the first piston.

### EXAMPLE 12c

The system of example 11c wherein: the first piston has a maximum range of motion within the first chamber; a plurality of steps by the step motor is needed to move the first piston across the maximum range of motion.

### EXAMPLE 13c

The system of example 2c wherein: the first piston has a first maximum range of motion within the first chamber; moving the first piston away from the filter, while the second piston is still, to advance the BMA into the first chamber from the first container includes moving the first piston away from the filter but not across an entirety of the first maximum range of motion; the at least one processor is to perform operations comprising, after moving the first piston away from the filter while the second piston is still, moving the second piston away from the filter while the first piston is still to advance the fluid fraction into the second chamber from the first container.

### EXAMPLE 14c

The system of example 13c wherein the filter simultaneously and directly contacts both of the first and second grates.

Thus, in an embodiment piston faces do not necessarily directly contact the grates.

### EXAMPLE 15c

The system of example 2c wherein the at least one processor is to perform operations comprising: move, at a first rate of speed, the first piston towards the filter while simultaneously moving the second piston away from the filter to advance the fluid fraction from the first chamber across the filter and into the second chamber and to locate the cellular fraction adjacent the filter and within the first chamber; move, at a second rate of speed, the first piston away from the filter while simultaneously moving the second piston towards the filter to advance the fluid fraction from the second chamber across the filter and into the first chamber and to flush the cellular fraction away from the filter; wherein the second rate of speed is faster than the first rate of speed.

### EXAMPLE 16c

The system of example 1c wherein: the first piston has a maximum range of motion within the first chamber, the maximum range of motion extending from a first area directly adjacent the filter to a second area at an opposite end of the maximum range of motion from the first area; a third area is between the first and second areas; the at least one processor is to perform operations comprising moving the first piston toward the filter starting from the third area and, as a result, moving the first piston toward the filter across less than an entirety of the maximum range of motion to advance the cellular component out of the first chamber and into the second container.

For example, in FIG. 5b the first area may be between portions of grate 507, the second area may be within the voids of the piston 501, and the third area may be the area between the first and second areas. A goal of an embodiment is produce a smaller volume of concentrated cellular fraction so during ejection of cellular fraction the piston 501 may start from an area that is less than the maximum pulled back position of the piston and instead start closer to the filter and make only a short push to expel a cell rich volume into an awaiting syringe or other such container.

### EXAMPLE 17c

The system of example 1c, wherein: the system does not include a centrifuge; the cellular fraction includes at least one of mononucleated cells, red blood cells, or platelets.

### EXAMPLE 18c

A blood filtering system comprising: a lower piston included in a lower chamber and an upper piston included in an upper chamber; a filter between the lower and upper pistons; at least one processor, coupled to at least one memory, to perform operations comprising: move the lower piston away from the filter, while the upper piston is still, to create a pressure differential that draws bone marrow aspirate (BMA) into the lower chamber from an external container; move the lower piston towards the filter while simultaneously moving the upper piston away from the filter to advance BMA fluid of the BMA from the lower chamber across the filter and into the upper chamber and to locate BMA cells of the BMA adjacent the filter and within the lower chamber; move the lower piston toward the filter, while the upper piston is still, to advance the BMA cells out of the lower chamber and into one of the external container and another external container.

Thus, embodiments may include pistons that do not necessarily have protrusions and voids and filter support grates may not be needed. For example, the piston faces may be flat or planar. Further, not all embodiments require a backflush operation. For example, the lower piston may slowly advance towards the filter. Gravity will pull the cellular fraction toward the piston face. The piston face may slowly proceed towards the filter as the upper piston moves away from the filter to allow filtration of the fluid fraction. However, at some point the upper piston may halt while the lower piston continues towards the filter. This may eject the contents remaining in the lower chamber whereby those contents may include a high concentration of the cellular fraction.

### EXAMPLE 19c

The system of example 18c wherein the at least one processor is to perform operations comprising move the lower piston away from the filter while simultaneously moving the upper piston towards the filter to advance the BMA fluid from the upper chamber across the filter and into the lower chamber and to flush the BMA cells away from the filter.

### EXAMPLE 20c

The system of example 18c comprising: a support member coupled to the filter to support the filter when the BMA fluid passes across the filter; and wherein the at least one processor is to perform operations comprising: move, at a first speed, the lower piston towards the filter; after moving the lower piston towards the filter at the first speed, move, at a second speed, the lower piston towards the filter while the upper piston is still to advance the BMA cells out of the lower chamber and into the one of the external container and the another external container; the first speed is faster than the second speed.

Embodiments have largely covered systems with at least one processor and at least one memory. However, other embodiments may include an entirely mechanical analog to such systems. For example, mechanical lever and gear systems may translate the pistons in a manner similar to that of the processor/memory/software/firmware embodiments. For instance, pulling a first lever may perform the intake phase (e.g., FIG. 5b), pulling a second lever may perform the filter phase (e.g., FIG. 6b), pulling a third lever may perform the backflush phase (e.g., FIG. 7b), and pulling a fourth lever may perform the eject phase (e.g., FIG. 8b).

Embodiments may include kits. For example, a kit may include the system of FIG. 1 along with various filters. The filters may include varying pore sizes so a user may select a filter based on whether RBCs, MNCs, and/or platelets are desired. Kits may include syringes such as the first and second containers of Example 1c.

### EXAMPLE 21c

A blood filtering system comprising: a first projection means included in a first chamber; a second projection means included in a second chamber; a filter means between the first and second projection means; a means to perform operations comprising: move the first piston away from the filter, while the second piston is still, to advance bone marrow aspirate (BMA) into the first chamber from a first container; move the first piston towards the filter while simultaneously moving the second piston away from the filter to: (a)(i) advance a fluid fraction of the BMA from the first chamber across the filter and into the second chamber, and (a)(ii) locate a cellular fraction of the BMA adjacent the filter and within the first chamber; move the first piston away from the filter while simultaneously moving the second piston towards the filter to advance the fluid fraction from the second chamber across the filter and into the first chamber and to flush the cellular fraction away from the filter; move the first piston toward the filter, while the second piston is still, to advance the cellular fraction out of the first chamber and into a second container.

### EXAMPLE 22c

A blood filtering system comprising: a first piston included in a first chamber; a second piston included in a second chamber; a filter between the first and second pistons; a means to move the first piston away from the filter, while the second piston is still, to advance bone marrow aspirate (BMA) into the first chamber from a first container; a means to move the first piston towards the filter while simultaneously moving the second piston away from the filter to: (a)(i) advance a fluid fraction of the BMA from the first chamber across the filter and into the second chamber, and (a)(ii) locate a cellular fraction of the BMA adjacent the filter and within the first chamber; a means to move the first piston away from the filter while simultaneously moving the second piston towards the filter to advance the fluid fraction from the second chamber across the filter and into the first chamber and to flush the cellular fraction away from the filter; a means to move the first piston toward the filter, while the second piston is still, to advance the cellular fraction out of the first chamber and into a second container.

### EXAMPLE 23c

The system of example 22c comprising: a first means to support the filter; and a second means to support the filter.

### EXAMPLE 24c

The system of example 23c comprising: means to move, at a first rate of speed, the first piston towards the filter but before the first piston is within a void of the first means to support the filer; means to move, at a second rate of speed, the first piston towards the filter while the first piston is within the void; wherein the second rate of speed is faster than the first rate of speed.

### EXAMPLE 25c

The system of example 22c comprising means to move the first piston at a first speed and at a second speed wherein the first speed is not equal to the second speed.

The foregoing description of the embodiments of the invention has been presented for the purposes of illustration and description. It is not intended to be exhaustive or to limit the invention to the precise forms disclosed. This description and the claims following include terms, such as left, right, top, bottom, over, under, upper, lower, first, second, etc. that are used for descriptive purposes only and are not to be construed as limiting. The term “on” as used herein (including in the claims) does not indicate that, for example, a first element “on” a second element is directly on and in immediate contact with the second element unless such is specifically stated; there may be a third element or other structure between the first element and the second element on the first element. The embodiments of a device or article described herein can be manufactured, used, or shipped in a number of positions and orientations. Persons skilled in the relevant art can appreciate that many modifications and variations are possible in light of the above teaching. Persons skilled in the art will recognize various equivalent combinations and substitutions for various components shown in the Figures. It is therefore intended that the scope of the invention be limited not by this detailed description, but rather by the claims appended hereto.

What is claimed is:

1. A blood filtering system comprising:
a first piston included in a first chamber;
a second piston included in a second chamber;
a filter between the first and second pistons;
at least one memory and at least one processor, coupled to the at least one memory, to perform operations comprising:
move the first piston away from the filter, while the second piston is still, to advance bone marrow aspirate (BMA) into the first chamber from a first container;
move the first piston towards the filter while simultaneously moving the second piston away from the filter to: (a)(i) advance a fluid fraction of the BMA from the first chamber across the filter and into the second chamber, and (a)(ii) locate a cellular fraction of the BMA adjacent the filter and within the first chamber;
move the first piston away from the filter while simultaneously moving the second piston towards the filter to advance the fluid fraction from the second chamber across the filter and into the first chamber and to flush the cellular fraction away from the filter;
move the first piston toward the filter, while the second piston is still, to advance the cellular fraction out of the first chamber and into a second container.

2. The system of claim 1 comprising:
a first grate, between the filter and the first piston, to support the filter; and
a second grate, between the filter and the second piston, to support the filter;
wherein the first grate has first grate protrusions arranged in a first grate pattern;
wherein the second grate has second grate protrusions arranged in a second grate pattern.

3. The system of claim 2 wherein:
the first piston has a first maximum range of motion within the first chamber;
the second piston has a second maximum range of motion within the second chamber;
the first piston has a first piston face having first piston protrusions arranged in a first piston pattern so the first piston protrusions fit between the first grate protrusions when the first piston is at one end of the first maximum range of motion;
the second piston has a second piston face having second piston protrusions arranged in a second piston pattern so the second piston protrusions fit between the second grate protrusions when the second piston is at one end of the second maximum range of motion.

4. The system of claim 3 wherein:
the first piston face has first voids between the first piston protrusions;
a first void of the first voids includes a first aperture;
the first aperture is coupled to a first channel that is to fluidly couple to the second container.

5. The system of claim 4 comprising a second aperture, wherein:
a second void of the first voids includes a second aperture;
the second aperture is coupled to a second channel that is to fluidly couple to the second container.

6. The system of claim 3 wherein:
the filter is substantially located in a plane;
one of the first grate protrusions extends away from the filter, in a direction orthogonal to the plane, a first distance;
the one of the first grate protrusions and another of the first grate protrusions define a void that extends away from the filter the first distance;
the at least one processor is to perform operations comprising:
move, at a first rate of speed, the first piston towards the filter but before the first piston is included in the void;
move, at a second rate of speed, the first piston towards the filter while the first piston is included in the void;
wherein the second rate of speed is faster than the first rate of speed.

7. The system of claim 3 comprising a first conduit, wherein:
the first conduit is to couple the first container to the first chamber;
the first conduit includes a first output located on the first piston face.

8. The system of claim 7 comprising a second conduit, wherein:
the second conduit is to couple the first container to the first chamber;
the second conduit includes a second output located on the first piston face.

9. The system of claim 2 comprising:
at least one motor coupled to the at least one processor;
wherein the at least one motor is to move the first piston within the first chamber in response to first commands from the at least one processor;
wherein the at least one motor is to move the second piston within the second chamber in response to second commands from the at least one processor.

10. The system of claim 9 wherein the at least one motor is move the first piston independently of moving the second piston.

11. The system of claim 9 wherein:
the at least one motor includes a step motor;
the at least one processor is to perform operations comprising:
convert a number of steps to be taken by the step motor to at least one of a volume or a flow rate; and
track a number of steps taken by the step motor to determine a location of the first piston.

12. The system of claim 11 wherein:
the first piston has a maximum range of motion within the first chamber;
a plurality of steps by the step motor is needed to move the first piston across the maximum range of motion.

13. The system of claim 2 wherein:
the first piston has a first maximum range of motion within the first chamber;
moving the first piston away from the filter, while the second piston is still, to advance the BMA into the first chamber from the first container includes moving the first piston away from the filter but not across an entirety of the first maximum range of motion;
the at least one processor is to perform operations comprising, after moving the first piston away from the filter while the second piston is still, moving the second piston away from the filter while the first piston is still to advance the fluid fraction into the second chamber from the first container.

14. The system of claim 13 wherein the filter simultaneously and directly contacts both of the first and second grates.

15. The system of claim 2 wherein the at least one processor is to perform operations comprising:
move, at a first rate of speed, the first piston towards the filter while simultaneously moving the second piston away from the filter to advance the fluid fraction from the first chamber across the filter and into the second chamber and to locate the cellular fraction adjacent the filter and within the first chamber;
move, at a second rate of speed, the first piston away from the filter while simultaneously moving the second piston towards the filter to advance the fluid fraction from the second chamber across the filter and into the first chamber and to flush the cellular fraction away from the filter;
wherein the second rate of speed is faster than the first rate of speed.

16. The system of claim 1 wherein:
the first piston has a maximum range of motion within the first chamber, the maximum range of motion extending from a first area directly adjacent the filter to a second area at an opposite end of the maximum range of motion from the first area;
a third area is between the first and second areas;
the at least one processor is to perform operations comprising moving the first piston toward the filter starting from the third area and, as a result, moving the first piston toward the filter across less than an entirety of the maximum range of motion to advance the cellular component out of the first chamber and into the second container.

17. The system of claim 1, wherein:
the system does not include a centrifuge;
the cellular fraction includes at least one of mononucleated cells, red blood cells, or platelets.

18. A blood filtering system comprising:
a lower piston included in a lower chamber and an upper piston included in an upper chamber;
a filter between the lower and upper pistons;
at least one processor, coupled to at least one memory, to perform operations comprising:
move the lower piston away from the filter, while the upper piston is still, to create a pressure differential that draws bone marrow aspirate (BMA) into the lower chamber from an external container;
move the lower piston towards the filter while simultaneously moving the upper piston away from the filter to advance BMA fluid of the BMA from the lower chamber across the filter and into the upper chamber and to locate BMA cells of the BMA adjacent the filter and within the lower chamber;
move the lower piston toward the filter, while the upper piston is still, to advance the BMA cells out of the lower chamber and into one of the external container and another external container.

19. The system of claim 18 wherein the at least one processor is to perform operations comprising move the lower piston away from the filter while simultaneously moving the upper piston towards the filter to advance the BMA fluid from the upper chamber across the filter and into the lower chamber and to flush the BMA cells away from the filter.

20. The system of claim 18 comprising:
a support member coupled to the filter to support the filter when the BMA fluid passes across the filter; and
wherein the at least one processor is to perform operations comprising:
move, at a first speed, the lower piston towards the filter;
after moving the lower piston towards the filter at the first speed, move, at a second speed, the lower piston towards the filter while the upper piston is still to advance the BMA cells out of the lower chamber and into the one of the external container and the another external container;
the first speed is faster than the second speed.
